(19) United States
(12) Patent Application Publication
(10) Pub. No.: US 2021/0315992 A1
(43) Pub. Date: Oct. 14, 2021

(54) ADJUVANT EFFECT OF THE TLR1/2 AGONIST DIPROVOCIM SYNERGIZES WITH CHECKPOINT-INHIBITING ANTIBODIES TO ELIMINATE DISEASE

(71) Applicant: The Board of Regents of the University of Texas System, Austin, TX (US)

(72) Inventors: Bruce Beutler, lrving, TX (US); Ying Wang, Dallas, TX (US)

(21) Appl. No.: 17/265,333

(22) PCT Filed: Jul. 31, 2019
(86) PCT No.: PCT/US2019/044421

§ 371 Date: Feb. 2, 2021

Related U.S. Application Data
(60) Provisional application No. 62/713,823, filed on Aug. 2, 2018.

(51) Int. Cl. A61K 39/39; A61K 39/00; A61K 39/015; A61K 39/145; A61P 35/00

(52) U.S. Cl. CPC A61K 39/39; A61K 2039/55511; A61K 39/001124; A61K 39/00118; A61K 39/001166; A61K 39/001106; A61K 39/001121; A61K 39/001131; A61K 39/001129; A61K 39/001174; A61K 39/001159; A61K 39/015; A61K 39/145; A61P 35/00; A61K 39/001118; A61K 39/001158; A61K 39/001128

(57) ABSTRACT

A potent human and mouse Toll-like receptor (TLR)1/TLR2 agonist was identified and optimized, Diprovocim, which exhibited an EC₅₀ of 110 pM in human THP-1 cells and 1.3 nM in primary mouse peritoneal macrophages. In mice, Diprovocim-adjuvanted ovalbumin immunization promoted antigen-specific humoral and CTL responses, and synergized with anti-PD-L1 treatment to inhibit tumor growth, generating long-term anti-tumor memory, curing or prolonging survival of mice engrafted with the murine melanoma B16-OVA. Diprovocim induced greater frequencies of tumor infiltrating leukocytes than alum, of which CD8 T cells were necessary for the antitumor effect of immunization plus anti-PD-L1 treatment.

### GOVERNMENTAL SUPPORT

This invention was made with governmental support under AI125581 awarded by the National Institutes of Health. The government has certain rights in the invention.

### BACKGROUND ART

By activating antigen-presenting cells (APCs) including dendritic cells (DCs) and macrophages, adjuvants hold the potential to unleash the natural functions of cytotoxic T lymphocytes (CTLs) to kill pathogens or cancer. Many adjuvants including TLR agonists engage innate immune receptors on APCs, inducing APCs to present antigens, produce cytokines, and provide costimulatory signals (1, 2) to antigen-specific CD8 T cells. In response to these signals, CD8 T cells proliferate and differentiate into CTLs capable of killing infected or tumor cells expressing their target antigen. In addition, such signals activate CD4 T cells, inducing their expansion and differentiation into Th1 or Th2 T helper cells (3).

One of the most important targets of improved adjuvant technology lies in the field of cancer immunotherapy, where the adaptive immune system is exploited to kill cancer cells based on their expression of cancer associated antigens or neo-antigens (4, 5). The effectiveness of cancer immunotherapy depends on the generation and activation of tumor-specific CTLs (5, 6) and on their maintenance of activity in vivo, leading to killing of tumor cells and a long lasting anti-tumor memory response (5). Thus, immune checkpoint inhibitors such as anti-PD-1, anti-PD-L1 and anti-CTLA-4 have achieved remarkable clinical success in the treatment of melanoma and other cancers through their action in blocking pathways that inhibit CTL activation (7, 8).

However, even among those tumors known to be susceptible to checkpoint blockade, response rates of only about 20% have been reported for PD-1/PD-L1 antibody treatment (5, 9), possibly due to insufficient numbers or activation of tumor-reactive CTLs, or their failure to infiltrate tumors. These deficiencies may be exacerbated by immunosuppression induced by the cancer environment.

In addition, the lack of T cell support is thought to be at least in part responsible for the lack of vigor exhibited by many synthetic vaccines against various pathogens as well as cancer-related cell surface antigens.

TLR ligands have long been known to act as adjuvants in adaptive immune responses (10, 11) signaling via adapter proteins (MyD88, TRIF, TRAM, MAL), kinases, and ubiquitin ligases to activate NF-κB and IRFs (12-14). These and other transcription factors induce the expression of thousands of genes that carry out the innate immune response (15). Several nucleotide-based adjuvants such as TLR3 agonist poly I:C (9), TLR9 agonist CpG (16), and STING agonist cGAMP (6) have been reported to improve the efficacy of immune checkpoint inhibitors in pre-clinical models for cancer treatment.

These approaches aim to increase the number of tumor-specific CTLs upon which checkpoint inhibitors can act. However, they have relied chiefly on natural TLR ligands, which are difficult to synthesize, and in some instances quite toxic, presumably because they become widely disseminated in vivo, and activate myeloid cells indiscriminately, producing cytokine storm (17). Development was sought of agonists with superior pharmacologic properties, with defined structural and molecular mechanisms from which key adjuvant design principles can be learned.

By screening a library of synthetic compounds, a potent human- and mouse-active TLR1/2 agonist, Diprovocim, was identified that has no structural similarity to any microbial TLR agonist. As described below, Diprovocim illustratively elicits strong adjuvant activity in mice, successfully inhibiting tumor growth and prolonging survival when combined with a cancer antigen and immune checkpoint blockade in the B16-OVA melanoma model.

### BRIEF SUMMARY OF THE INVENTION

Adjuvants enhance adaptive immune responses, sometimes through unknown mechanisms, and can be used to augment both humoral and cellular responses to cancer antigens. The invention described herein illustrates synergistic immunological effects of the synthetic chemical adjuvant Diprovocim, which targets the innate immune receptor TLR2/TLR1 in mice and humans, that when used in conjunction with a diseased cell marker immunogen molecule and a checkpoint inhibitor provides a synergistic effect in inhibiting the growth of a diseased cell as compared to the use of the individual components or any two of those components.

The present invention thus contemplates a method of inhibiting the growth of diseased cells in a mammal. Those diseased cells express a marker molecule that is absent from cells of the same type that are free of the disease or is present in the disease-free cells in significantly reduced numbers compared to said diseased cells. A contemplated method comprises the steps of immunizing a diseased mammal in need by: a) administering to the diseased mammal (i) an adjuvant-sufficient amount of a Diprovocim compound, (ii) a T cell-stimulating amount of an immune checkpoint inhibitor, and (iii) an immunizing amount of a disease marker molecule. The immunized mammal is b) maintained for a time period sufficient for the mammal to mount an immune response to the immunization and inhibit growth of diseased cells.

A contemplated Diprovocim compound corresponds in structure to structural Formula V,

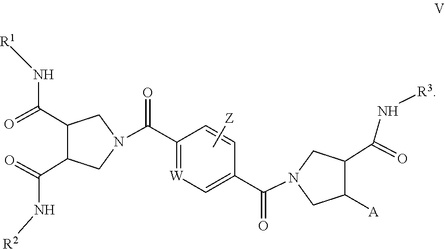

wherein
-A is —H (hydrido) or —C(O)NH—R⁴;
R¹, R², R³ and R⁴ are the same or different and are a 2-(4-fluorophenyl)ethyl, a trans-2-phenylcyclopropyl, a trans-2-(4-fluorophenyl)-cyclopropyl or a C₂-C₁₈ hydrocarbyl group with the provisos that:
1) at least two of R¹, R², R³ and R⁴ (R¹⁻⁴) or at least two of R¹, R², and R³ (R¹⁻³) are a trans-2-phenylcyclopropyl, a trans-2-(4-fluorophenyl)-cyclopropyl group or a mixture thereof, or each of R¹⁻⁴ is a 2-(4-fluorophenyl)ethyl group,
2) at least one depicted pyrrolidinyldicarboxamido group has the (S,S) configuration, and each depicted R substituent other than a C₂-C₁₈ hydrocarbyl group is a trans-2-phenylcyclopropyl, a trans-2-(4-fluorophenyl)-cyclopropyl group or a mixture thereof when each of R¹⁻⁴ is other than 2-(4-fluorophenyl)ethyl,
3) no more than two of R¹⁻⁴ are C₂-C₁₈ hydrocarbyl groups when -A is —C(O)NH—R⁴, and
4) when A is hydrido, one of R¹⁻³ can be a C₂-C₁₈ hydrocarbyl group and the depicted R³-containing pyrrolidinylcarboxamido group can have either the R or S configurations, or a mixture of both configurations;
—Z is one or more of halogen —H, —NH₂, —OH, —OCH₃, —NO₂, —OCH₂CO₂H, —O(CH₂CH₂O)ₙCH₂CH₂CO₂H, —OCH₂CONH(CH₂CH₂O)ₙCH₂CH₂CO₂H, —NHCOCH₂O—(CH₂CH₂O)ₙCH₂CO₂H, —OCH₂CONHCH₂CONHCH(CHOH)CO₂H, —OCH₂CONHCH₂CONHCHCO₂H(CH₂CO₂H), —OCH₂CONHCH₂CONHCH(CHOH) (CH₂CH₂O)ₙCH₂CH₂CO₂H, —OCH₂CONHCH₂CONHCH(CHOH) (CH₂CH₂O)ₙCH₂CH₂CO₂H, —OCH₂CONHCH₂CONHCH[(CH₂)₄NH₂]CO₂H, —OCH₂CONHCH₂CONHCH(CH₂OH)CO{NHCH[(CH₂)₄NH₂]CO}ₘNHCH—[(CH₂)₄NH₂]CO₂H (SEQ ID NOs: 3-8), —OCH₂CONHCH₂CO{NHCH[(CH₂)₄NH₂]CO}ₚNHCH[(CH₂)₄NH]CO₂H (SEQ ID NOs: 9-13) and —OCH₂CONHCH₂CO{NHCH(CH₂OH)CO}_{q}NHCH(CH₂OH)CO₂H (SEQ ID NOs: 14-18);
W is nitrogen (N) or CH;
“n” is a number whose average value is one to about eight;
“m” is a number whose value is 1 to about 6; “p” is a number whose value is 1 to about 6; and
“q” is a number whose value is 1 to about 6.

Diprovocim-1 (WO 2018/005812; Compound 3) displayed strong adjuvant activity in mice, particularly abetting cellular immune responses.

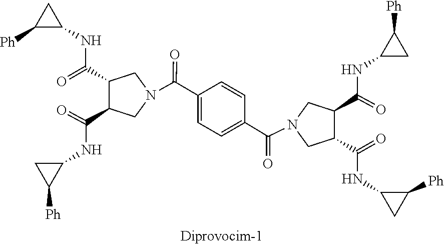 [SMILES: O=C(N[C@H]1C[C@@H]1c1ccccc1)[C@@H]1CN(C(=O)c2ccc(C(=O)N3C[C@@H](C(=O)N[C@H]4C[C@@H]4c4ccccc4)[C@H](C(=O)N[C@H]4C[C@@H]4c4ccccc4)C3)cc2)C[C@H]1C(=O)N[C@H]1C[C@@H]1c1ccccc1]

Immunization against a genetically engineered tumor-specific antigen, ovalbumin, when adjuvanted with Diprovocim-1, inhibited the growth of B16 melanoma and prolonged survival in the presence of immune checkpoint blockade by anti-PD-L1. Immunization with ovalbumin plus the Diprovocim or ovalbumin plus the anti-PD-L1 inhibitor, exhibited some benefit, but 100% of mice responded to immunization with the three components: Diprovocim, immunogen and checkpoint inhibitor. The data suggest Diprovocim-1 boosts the success of anti-PD-L1 treatment by increasing the number and activation of tumor-specific CTLs capable of responding to this checkpoint inhibitor.

As disclosed in WO 2018/005812 and Morin et al., J Am Chem Soc, In Press (2018), several members of the Diprovocim family have been prepared, their activity assayed, and have been given numbers such as Diprovocim-1 through Diprovocim-6. Several other compounds of the Diprovocim family having similar activity profiles, similar to somewhat lesser activity values in the assays used, have also been prepared and assayed.

Diprovocim-1 has been used herein as an exemplary member of the whole family, and is to be understood hereinafter to mean a Diprovocim family member is used when the word Diprovocim is used without an added hyphenated numeral. Thus, when used in connection with the words “family” or “a” as in “Diprovocim family” or “a Diprovocim”, the word “Diprovocim” is used to mean a member of the Diprovocim family as defined by Formula V. The word “Diprovocim” used with the word “preferred” refers to a compound of Formula I, and the phrase “more preferred” or “more preferably” refers to a Diprovocim family compound of Formula Ia. A “most preferred” Diprovocim is one of the compounds lettered A-I noted below.

A preferred member of the Diprovocim family of compounds is a compound that corresponds in structure to structural Formula I, below,

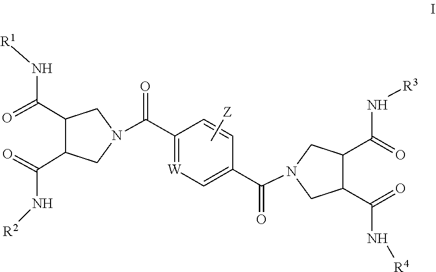

in which -A -C(O)NH—R⁴. The depicted R¹⁻⁴, W and Z moieties are as described above.

A still more preferred member of the Diprovocim family of compounds is a compound that corresponds in structure to structural Formula Ia, below,

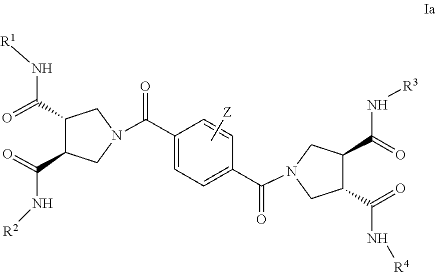

Structural formulas of currently most preferred compounds of Formula Ia in which R¹⁻⁴ are as previously defined, and —Z is hydrido (—H) are shown hereinafter as compounds of Formulas A, B, C, D, E, F, G, H, and I.

A Diprovocim is utilized in an adjuvant-sufficient amount to contact host mammal cells that express an antigenic disease-related marker molecule.

The host mammal cells that express an antigenic disease-related marker compound such as a peptide sequence are also contacted with an immune response-stimulating amount of a checkpoint inhibitor, preferably an antibody or paratope-containing antibody portion.

### BRIEF DESCRIPTION OF THE DRAWINGS

In the drawings forming a portion of this disclosure

FIG. 1A through FIG. 1F provide a series of graphs that illustrate that Diprovocim induces cytokine secretion by mouse and human cells. FIG. 1A-1D illustrates amounts of TNF in the supernatants of human THP-1 cells (FIG. 1A), human PBMC (FIG. 1B), mouse peritoneal macrophages (FIG. 1C), or mouse BMDC (FIG. 1D) after treatment with Diprovocim-1 for 4 hours (FIG. 1A, FIGS. 1C-1D) or 24 hours (FIG. 1B). FIG. 1E illustrates the amounts of IL-6 in the supernatants of mouse BMDC after treatment with Diprovocim for 4 hours. In each of FIG. 1A-1E the means of three independent samples are plotted; P values were determined by one-way ANOVA to compare the responses to different doses; in all studies P<0.0001. Results in FIG. 1A-FIG. 1E are representative of two independent studies.

FIG. 2A through FIG. 2D illustrate that a Diprovocim activates mouse and human TLR1/TLR2. FIG. 2A is a graph showing the amount of TNF in the supernatants of mouse peritoneal macrophages of the indicated genotypes after treatment with Diprovocim (500 nM) for 4 hours (n=3 mice per genotype). Cytokine levels were normalized to those of stimulated C57BL/6J cells. P values were determined by Student's t test and represent the significance of differences between responses of stimulated C57BL/6J cells and stimulated cells of mutant genotypes; four stars indicate those with statistically significant differences. FIG. 2B is a graph showing the amount of TNF in the supernatants of human THP-1 cells pretreated with control antibody, anti-TLR1 (20 μg/ml), or anti-TLR2 (20 μg/ml) for 1 hour, followed by treatment with vehicle or Diprovocim (250 pM) for another 4 hours. P values were determined by Student's t test. In FIG. 2A and FIG. 2B the means of three independent samples are plotted. FIG. 2C and FIG. 2D are immunoblot show the results of analysis of lysates of human THP-1 cells (FIG. 2C) and mouse peritoneal macrophages (FIG. 2D) treated with Diprovocim (5 nM in THP-1 and 500 nM in mouse peritoneal macrophages) for the indicated times. All results are representative of two independent studies.

FIG. 3A through FIG. 3G illustrate through graphs and schemes that Diprovocim enhances antigen-specific antibody and CTL responses. FIG. 3A-FIG. 3C show results WT or Tlr2^{-/-} C57BL/6J mice (4 mice per group) were immunized i.m. with 100 μg OVA mixed with vehicle, Diprovocim (10 mg/kg), or alum (2 mg/kg). After 14 days, serum titers of OVA-specific IgG (FIG. 3A), OVA-specific IgG1 (FIG. 3B) and OVA-specific IgG2b (FIG. 3C) were measured by ELISA. FIG. 3D shows a schematic of the experimental setup (left) and results for detection (center) and quantification (right) of CD69 expression on OT-I CD8 T cells by flow cytometery after 24 hours co-culture with DC collected from mice 24 hours after they were immunized i.m. with OVA mixed with vehicle or Diprovocim (4 mice per group).

FIG. 3E and FIG. 3F show results from mice that were unimmunized or immunized i.m. with 100 μg OVA mixed with vehicle or Diprovocim (10 mg/kg) (4 mice per group). Seven days after immunization, mice were injected i.v. with Celltrace Violet-labeled mouse splenocytes that were unpulsed (control cells) or pulsed with OVA peptide (a.a. 257-263) (target cells). Two days later, blood was collected to measure remaining live dye-labeled cells. FIG. 3E illustrates representative flow cytometry plots that count remaining target cells (right peak) and control cells (left peak) in wild type mice. FIG. 3F shows a quantitative comparison of the percentage of target cells killed in WT, Tlr1^{-/-} or Tlr2^{-/-} mice. P values were determined by Student's t test. All results are representative of two independent studies.

FIG. 4A through FIG. 4J illustrate inhibition of B16-OVA tumor growth by pre- or post-tumor treatment with Diprovocim-adjuvanted tumor vaccination and checkpoint blockade. FIG. 4A provides a schematic of pre- and post-tumor treatment protocols. C57BL/6J mice (n=8 mice per treatment) were injected s.c. with 2×10⁵ B16-OVA melanoma cells on day 0. For pre-tumor treatment, mice were immunized i.m. with OVA (100 μg) mixed with vehicle or Diprovocim (10 mg/kg) or alum (2 mg/kg) on the same day prior to tumor injection. Mice received a booster immunization seven days later in which anti-PD-L1 (200 μg) was administered on days 3, 6 and 9 after tumor inoculation by i.p. injection. For post-tumor treatment, initial immunization was on day 3 after tumor inoculation, with a booster seven days later. Anti-PD-L1 (200 μg) was administered on days 3, 6, 9, 12, and 15 after tumor inoculation by i.p. injection. FIG. 4B through FIG. 4F illustrate pre-tumor treatment results. Tumor volume (FIG. 4B and FIG. 4D) and percent mouse survival (survivors/total mice) (FIG. 4C and FIG. 4E). FIG. 4F shows naïve mice (n=8) or day 35 tumor-free survivors (n=8) from (FIG. 4C) were challenged with 2×10⁵ cells each B16-OVA and B16F10 tumor cells by s.c. injection, and tumor volume was monitored. FIG. 4G through FIG. 4J illustrate a comparison of pre-tumor (FIG. 4G and FIG. 4H) vs. post-tumor treatment (FIG. 4I and FIG. 4J). Tumor volume (FIG. 4G and FIG. 4I) and percent mouse survival (survivors/total mice) (FIG. 4H and FIG. 4J) are shown. P values for tumor volume analysis apply to the final time point as indicated in graphs and were calculated by Student's t test. P values for survival analysis were calculated by Kaplan-Meier analysis. All results are representative of two independent studies.

FIG. 5A through FIG. 5M illustrate that a Diprovocim enhances TILs and anti-tumor CTL responses. FIG. 5A is a schematic of treatment protocol for FIG. 5A through FIG. 5J in which C57BL/6J mice (n=6 mice per treatment) were injected s.c. with 2×10⁵ B16-OVA melanoma cells on day 0 and three days later, immunized i.m. with OVA (100 μg) mixed with vehicle or Diprovocim (10 mg/kg) or alum (2 mg/kg). Mice received a booster immunization on day 10 after tumor inoculation in which anti-PD-L1 (200 μg) was administered on days 3, 6, 9, and 12 after tumor inoculation by i.p. injection. Tumors were harvested on day 14 to isolate and analyze tumor-infiltrating leukocytes (TILs). FIG. 5A through FIG. 5J illustrate the frequency of each cell type out of total tumor cells is shown. FIG. 5B shows TILs (CD45⁺). FIG. 5C shows CD4 T cells (CD4⁺CD3⁺CD45⁺). FIG. 5D shows activated CD4 T cells (CD44^{high}OCD4⁺CD3⁺CD45⁺). FIG. 5E shows CD8 T cells (CD8⁺CD3⁺CD45⁺). FIG. 5F shows activated CD8 T cells (CD44^{high}CD8⁺CD3⁺CD45⁺). FIG. 5G shows OVA-specific CD8 T cells bearing a T-cell receptor specific for OVA₍₂₅₇₋₂₆₄₎-H2Kb tetramer. FIG. 5H shows NK cells (NK1.1⁺CD3⁻CD45⁺). FIG. 5I shows DCs (CD11c⁺CD3⁻CD45⁺). FIG. 5J shows macrophages (F4/80⁺CD11b⁺CD45⁺).

FIG. 5K is a schematic of treatment a protocol for FIG. 5L and FIG. 5M in which C57BL/6J mice (n=8 mice per treatment) were injected s.c. with 2×10⁵ B16-OVA melanoma cells on day 0 and three days later immunized i.m. with OVA (100 μg) mixed with vehicle or Diprovocim (10 mg/kg). Mice received a booster immunization on day 10 after tumor inoculation in which anti-PD-L1 (200 μg) was administered on day 3, 6, 9, 12, and 15 after tumor inoculation by i.p. injection. On day 0, 3, 6, 9, 12, and 15, anti-CD4 (300 μg), anti-CD8 (300 μg), anti-NK1.1 (300 μg), or a mixture of these three antibodies was administered to C57BL/6J mice by i.p. injection. Tumor volume (FIG. 5L) and percent mouse survival (survivors/total mice) (FIG. 5M) are illustrated. P values for tumor volume analysis apply to the final time point as indicated in graphs and were calculated by Student's t test. P values for survival analysis were calculated by Kaplan-Meier analysis. All results are representative of two independent studies.

FIG. 6 shows a schematic model of key cellular events mediating the antitumor effect of Diprovocim-adjuvant immunization plus checkpoint inhibition.

FIG. 7 illustrates that a Diprovocim does not stimulate IFN-β secretion by mouse peritoneal macrophages. IFN-β in the supernatants of mouse peritoneal macrophages after treatment with Diprovocim or LPS for 4 hours was assayed. The means of three independent samples are plotted. P values were determined by Student's t test; no significant differences were found between responses of unstimulated cells (0 nM) and Diprovocim-stimulated cells. Results are representative of two independent studies.

FIG. 8A and FIG. 8B illustrate that anti-PD-L1 antibodies do not inhibit B16 tumor growth in mice. C57BL/6J mice (n=8) were injected s.c. with 2×10⁵ B16-OVA melanoma cells on day 0. Anti-PD-L1 (200 μg) or mouse IgG2a isotype control antibody was administered on days 3, 6 and 9 after tumor inoculation by i.p. injection. FIG. 8A is a graph of tumor volume versus time and FIG. 8B is a graph showing percent mouse survival (survivors/total mice) versus time. The control values in both plots are shown above those for the anti-PD-L1 values where the two values diverge. The P value for tumor volume analysis applies to the final time point and was calculated by Student's t test; no significant difference was found between treatments. P values for survival analysis were calculated by Kaplan-Meier analysis; no significant difference was found between treatments. Results are representative of two independent studies.

FIG. 9A and FIG. 9B illustrate that a Diprovocim is more potent than Pam₃CSK₄ in activation of TNF production in human cells. FIG. 9A and FIG. 9B show TNF amounts assayed from the supernatants of human THP-1 cells (FIG. 9A) and human PBMC (FIG. 9B) after treatment with Diprovocim or Pam₃CSK₄ for 4 hours (FIG. 9A) or 24 hours (FIG. 9B). Data points for Diprovocim are shown generally to the left of data points for Pam₃CSK₄ in FIG. 9A, above data points for Pam₃CSK₄ where the lines diverge in FIG. 9B. The means of three independent samples are plotted. Results are representative of two independent studies.

### DEFINITIONS

Antibody: a polypeptide that immunologically binds to a ligand group. Antibodies, as used herein, are immunoglobulin molecules and immunologically active fragments of immunoglobulin molecules. Such portions known in the art as Fab, Fab′; F(ab′)₂ and F_{V} are included. Typically, antibodies bind ligands that range in size from about 6 through about 34 Angstroms (Å) with association constants in the range of about 10⁴ to about 10¹⁰ M⁻¹, and as high as 10¹³ M⁻¹. Antibodies can bind a wide range of ligands, including small molecules such as steroids and prostaglandins, biopolymers such as nucleic acids, proteins and polysaccharides, and synthetic polymers such as polypropylene.

An “antibody combining site” or “paratope” is that structural portion of an antibody molecule comprised of a heavy and light chain variable and hypervariable regions that specifically binds to (immunoreacts with) an “antigen” or “epitope”.

The term “antibody” is meant to particularly encompass monoclonal antibodies that are suitable for injection (pharmaceutically acceptable) into a diseased mammal in need of treatment without undo adverse effects due to contaminants. Such monoclonal antibodies can be obtained from the animal species that is immunized as discussed herein, such as a human. Or, the antibodies can be induced in one animal and the antibody-producing cells modified to produce antibody protein sequences of the mammal to be immunized. Although other species of mammal are contemplated for immunization, a human is a particularly preferred recipient of the immunization. As a consequence, a contemplated monoclonal antibody that was originally induced in a mouse, for example, can be more useful to a human recipient as a so-called “humanized” antibody, or as a “chimeric” antibody. These terms are used herein as described in International Nonproprietary Names (INN) for biological and biotechnological substances (a review), World Health Organization (2016), § 2.7.

The word “antigen” has been used historically to designate an entity that is bound by an antibody or receptor, and also to designate the entity that induces the production of the antibody. More current usage limits the meaning of antigen to that entity bound by an antibody or receptor, whereas the word “immunogen” is used for the entity that induces antibody production or binds to the receptor. Where an entity discussed herein is both immunogenic and antigenic, reference to it as either an immunogen or antigen is typically made according to its intended utility.

The term “immunoreact” in its various forms is used herein to refer to specific binding between an antigenic determinant-containing molecule (antigen) and a molecule containing an antibody combining site such as a whole antibody molecule or a paratope-containing portion thereof.

An “antigenic determinant” is the structural portion of the antigen that is immunologically bound by an antibody combining site or T cell receptor. The term is also used interchangeably with “epitope”. Antibodies can bind a single epitope of an antigen (monoclonal) or multiple epitopes (polyclonal). In a proteinaceous material, the length of a linear epitope is usually recited as being about 5 to about 7 amino acid residues.

The articles “a” and “an” are used herein to refer to one or to more than one (i.e., to at least one) of the grammatical object of the article. By way of example, “an element” means one element or more than one element.

The word “hydrocarbyl” is used herein as a short hand term for a non-aromatic group that includes straight and branched chain aliphatic as well as alicyclic groups or radicals that contain only carbon and hydrogen. Thus, alkyl, alkenyl and alkynyl groups are contemplated, whereas aromatic hydrocarbons such as phenyl and naphthyl groups, which strictly speaking are also hydrocarbyl groups, are referred to herein as aryl groups or radicals, as discussed hereinafter.

Where a specific aliphatic hydrocarbyl substituent group is intended, that group is recited; i.e., methyl, ethyl, butyl, tert-butyl, hexyl, hexenyl, 2-ethylhexyl, dodecyl (C₁₂), octadecyl (C₁₈). A particularly preferred hydrocarbyl group is an alkyl group. As a consequence, a generalized, but more preferred substituent can be recited by replacing the descriptor “hydrocarbyl” with “alkyl” in any of the substituent groups enumerated herein.

Although long chain (e.g., C₁₈) hydrocarbyl groups are contemplated, examples of shorter (C₁-C₄) groups are used illustratively here. Such illustrative alkyl radicals include ethyl, n-propyl, isopropyl, n-butyl, isobutyl, sec-butyl, tert-butyl and cyclopropyl. Examples of suitable alkenyl radicals include ethenyl (vinyl), 2-propenyl, 3-propenyl, 1,4-butadienyl, 1-butenyl, 2-butenyl, and 3-butenyl. Examples of alkynyl radicals include ethynyl, 2-propynyl, 1-propynyl, 1-butynyl, 2-butynyl, 3-butynyl, and 1-methyl-2-propynyl.

As a skilled worker will understand, a substituent that cannot exist such as a C₁ alkenyl group is not intended to be encompassed by the word “hydrocarbyl”, although such substituents with two or more carbon atoms are intended.

Usual chemical suffix nomenclature is followed when using the word “hydrocarbyl” except that the usual practice of removing the terminal “yl” and adding an appropriate suffix is not always followed because of the possible similarity of a resulting name to one or more substituents. Thus, a hydrocarbyl ether is referred to as a “hydrocarbyloxy” group rather than a “hydrocarboxy” group as may possibly be more proper when following the usual rules of chemical nomenclature. Illustrative hydrocarbyloxy groups include methoxy, ethoxy, n-propoxy, isopropoxy, allyloxy, n-butoxy, iso-butoxy, sec-butoxy, and tert-butoxy groups.

Ligand: a molecule having a structural region that binds specifically to a particular receptor molecule, usually via electrostatic forces and/or hydrogen bonds. An exemplary ligand is the epidermal growth factor molecule.

The “marker molecule” (antigen or immunogen) can be but need not be expressed on the cell surface, and rather can be expressed anywhere in the diseased cell. The reason for that is that substantially all of the native cellular proteins of mammals are processed into shorter peptides by the cell and bound extracellularly by class I MHC molecules. Such native proteins are typically so processed during the organism's immaturity, and T cells or other immune cells that may be induced by those native protein portions are eliminated by the organism prior to maturity, resulting in “self protein” tolerance. As a result, except in cases of certain immunological diseases, only foreign peptides or neoantigenic peptides caused by disease such as cancer whose cells result from mutation are recognized as “foreign” and induce an immune response to the MHC-bound peptide.

Further, as to mutations, at times the mutation can be a frame-shift mutation, and an un-natural sequence of amino acids results. The marker molecule can also be a “tumor antigen;” that is, a protein that can be expressed by other cells during embryonic development, for example, but is characteristically expressed much more by tumors than by normal cells. Or the marker molecule can be an oncogene product, for example, an abnormal fusion protein created by a recombination event within tumor cells. The marker molecule can also be the product of an infectious agent such as a virus or bacterium as well.

Peptide/Polypeptide: an oligomer or polymer comprising at least two amino acid residues in which adjacent residues are linked by a peptide bond between the alpha-amino group of one residue and the alpha-carboxyl group of an adjacent residue. The primary structure of a polypeptide has a primary amine group at one terminus and a carboxylic acid group at the other terminus of the polymer. A peptide or polypeptide is depicted herein and usually in the art from left to right and in the direction from amino-terminus to carboxy-terminus. Also, a polypeptide in aqueous solution is usually in one or more zwitterionic forms depending on the pH of the solution. The words “peptide” and “polypeptide” are used interchangeably herein.

Protein: a single polypeptide or set of cross-linked polypeptides comprising more than about 100 amino acid residues. Proteins can have chemical crosslinking, e.g., via disulfide bridges, within the same polypeptide chain or between adjacent polypeptides. When a protein is glycosylated it can be called a glycoprotein. When a protein comprises one or more discrete polypeptide/protein subunits linked together, as by a peptide linkage, amino acid residue sequence, disulfide bridge, and the like, the protein is frequently termed a fusion protein, fusion polypeptide, chimeric fusion, and the like.

Receptor: a biologically active proteinaceous molecule having a structural region that specifically binds to (or with) another molecule (ligand). An exemplary receptor molecule is an antibody combining site or a transmembrane cellular protein molecule involved in intra- or intercellular signaling such as the endothelial growth hormone receptor referred to as EGFR, ERBB and also as HER2, and the like.

The term “residue” is used interchangeably with the phrase amino acid residue. All amino acid residues identified herein are in the natural or L-configuration. In keeping with standard polypeptide nomenclature, [J. Biol. Chem., 243:3557-59 (1969)], abbreviations for amino acid residues are as shown in the following Table of Correspondence.

TABLE OF CORRESPONDENCE

| 1-Letter | 3-Letter | AMINO ACID NAME |
|---|---|---|
| Y | Tyr | L-tyrosine |
| G | Gly | glycine |
| F | Phe | L-phenylalanine |
| M | Met | L-methionine |
| A | Ala | L-alanine |
| S | Ser | L-serine |
| I | Ile | L-isoleucine |
| L | Leu | L-leucine |
| T | Thr | L-threonine |
| V | Val | L-valine |
| P | Pro | L-proline |
| K | Lys | L-lysine |
| H | His | L-histidine |
| Q | Gln | L-glutamine |
| E | Glu | L-glutamic acid |
| Z | Glx | L-glutamic acid or L-glutamine |
| W | Trp | L-tryptophan |
| R | Arg | L-arginine |
| D | Asp | L-aspartic acid |
| N | Asn | L-asparagine |
| B | Asx | L-aspartic acid or L-asparagine |
| C | Cys | L-cysteine |

The present invention has several benefits and advantages.

A salient benefit of the invention is that the combination immunization provides synergistic results in inhibiting diseased cell growth.

An advantage of the invention is that the combination immunization provides T cell help that virus- and bacteria-free vaccines have often lacked.

Another benefit that the invention provides is that those skilled in the art have been finding, studying and publishing formulas of disease-related immunogens that have been ultimately unsuccessful since the early 1980's but can now be successfully put to use.

Still further benefits and advantages of the invention will be apparent to the skilled worker from the disclosures that follow.

### DETAILED DESCRIPTION OF PREFERRED EMBODIMENTS

The present invention contemplates a method of inhibiting the growth of diseased cells in a mammal. Those diseased cells express one or more marker molecules that are absent on cells of the same type that are free of the disease or are present in the disease-free cells in significantly reduced numbers compared to the diseased cells. Workers skilled in the art have published numerous articles and reviews discussing marker molecules that are present in diseased cells in amounts significantly greater than the amount present in disease-free cells and methods for determining those differences. See, for example, Kim et al., NIB Rep 50(6):285-298 (2017) and the citations therein. In addition, techniques such as quantitative western blots performed with optical density scans or radioactivity detection means and other means are well known in the art. Cells “of the same type” are disease-free cells from the same organ and tissue as the diseased cells.

A contemplated method comprises the steps of immunizing a diseased mammal in need by: a) administering to the diseased mammal (i) an adjuvant-sufficient amount of a Diprovocim compound, (ii) a T cell-stimulating amount of an immune checkpoint inhibitor, and (iii) an immunizing amount of that marker molecule. The immunized mammal is b) maintained for a time period sufficient for the mammal to mount an immune response to the immunization and inhibit growth of diseased cells.

A contemplated Diprovocim compound corresponds in structure to structural Formula V,

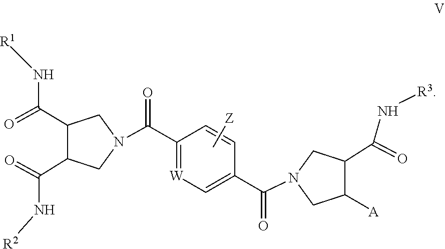

wherein
-A is —H (hydrido) or —C(O)NH—R⁴;
R¹, R², R³ and R⁴ are the same or different and are a 2-(4-fluorophenyl)ethyl, a trans-2-phenylcyclopropyl, a trans-2-(4-fluorophenyl)-cyclopropyl or a C₂-C₁₈ hydrocarbyl group with the provisos that:
1) at least two of R¹, R², R³ and R⁴ (R¹⁻⁴) or at least two of R¹, R², and R³ (R¹⁻³) are a trans-2-phenylcyclopropyl, a trans-2-(4-fluorophenyl)-cyclopropyl group or a mixture thereof, or each of R¹⁻⁴ is a 2-(4-fluorophenyl)ethyl group,
2) at least one depicted pyrrolidinyldicarboxamido group has the (S,S) configuration, and each depicted R substituent other than a C₂-C₁₈ hydrocarbyl group is a trans-2-phenylcyclopropyl, a trans-2-(4-fluorophenyl)-cyclopropyl group or a mixture thereof when each of R¹⁻⁴ is other than 2-(4-fluorophenyl)ethyl,
3) no more than two of R¹⁻⁴ are C₂-C₁₈ hydrocarbyl groups when -A is —C(O)NH—R⁴, and
4) when A is hydrido, one of R¹⁻³ can be a C₂-C₁₈ hydrocarbyl group and the depicted R³-containing pyrrolidinylcarboxamido group can have either the R or S configurations, or a mixture of both configurations;
—Z is one or more of halogen —H, —NH₂, —OH, —OCH₃, —NO₂, —OCH₂CO₂H, —O(CH₂CH₂O)ₙCH₂CH₂CO₂H, —OCH₂CONH(CH₂CH₂O)ₙCH₂CH₂CO₂H, —NHCOCH₂O—(CH₂CH₂O)ₙCH₂CO₂H, —OCH₂CONHCH₂CONHCH(CHOH)CO₂H, —OCH₂CONHCH₂CONHCHCO₂H(CH₂CO₂H), —OCH₂CONHCH₂CONHCH(CHOH) (CH₂CH₂O)ₙCH₂CH₂CO₂H, —OCH₂CONHCH₂CONHCH(CHOH) (CH₂CH₂O)ₙCH₂CH₂CO₂H, —OCH₂CONHCH₂CONHCH[(CH₂)₄NH₂]CO₂H, —OCH₂CONHCH₂CONHCH(CH₂OH)CO{NHCH[(CH₂)₄NH₂]CO}ₘNHCH—[(CH₂)₄NH₂]CO₂H (SEQ ID NOs: 3-8), —OCH₂CONHCH₂CO{NHCH[(CH₂)₄NH₂]CO}ₚNHCH[(CH₂)₄NH]CO₂H (SEQ ID NOs: 9-13) and —OCH₂CONHCH₂CO{NHCH(CH₂OH)CO}_{q}NHCH(CH₂OH)CO₂H (SEQ ID NOs: 14-18);
W is nitrogen (N) or CH;
“n” is a number whose average value is one to about eight;
“m” is a number whose value is 1 to about 6; “p” is a number whose value is 1 to about 6; and
“q” is a number whose value is 1 to about 6.

A preferred member of the Diprovocim family of compounds of Formula V is a compound that corresponds in structure to structural Formula I, below, in which -A=-C(O)NH—R⁴, and the depicted

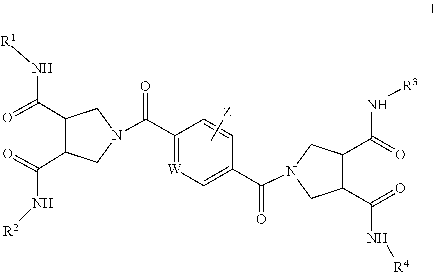

R¹⁻⁴, W and Z moieties are as described above.

In one more preferred compound of Formula I, at least one member of substituent pair R¹ and R³ (either R¹ or R³) or pair R¹ and R² (either R¹ or R²) is a trans-2-phenylcyclopropyl or trans-2-(4-fluorophenyl)cyclopropyl group. It is also preferred that at least one member of substituent pair R¹ and R³ or pair R¹ and R² has the (1S,2R) configuration of a trans-2-phenylcyclopropyl or trans-2-(4-fluoro-phenyl)cyclopropyl group.

More preferably, at least three substituents of R¹, R², R³ and R⁴ have the (1S,2R) configuration of a trans-2-phenylcyclopropyl or trans-2-(4-fluorophenyl)cyclopropyl group. More preferably still, each of R¹, R², R³ and R⁴ has the (1S,2R) configuration of a trans-2-phenylcyclopropyl or a trans-2-(4-fluorophenyl)cyclopropyl group.

In another preference, each depicted pyrrolidinyldicarboxamido group has the (S,S) configuration and each depicted R¹⁻⁴ substituent is a trans-2-phenylcyclopropyl, a trans-2-(4-fluoro-phenyl)cyclopropyl group or a mixture thereof, and bonds to the cyclopropyl moiety have a (1S,2R) configuration.

In another aspect, up to two of R¹⁻⁴ in a compound of Formula I or one of its sub-generic formulas can be a C₂-C₁₈ hydrocarbyl group. It is preferable that the hydrocarbyl group be an alkyl group and have a length of 4 to about 16 carbon atoms, and more preferably still, about 6 to about 10 carbon atoms. Straight chained hydrocarbyl groups are also preferred, although up to two methyl and ethyl group substituents or both can be present as can an carbocyclic ring, and also one or two double or triple bonds. Specific C₂-C₁₈ hydrocarbyl groups are discussed previously in the discussion of the use of the word hydrocarbyl.

As is seen from Formula I, each molecule contains at least one, and preferably two, 3,4-pyrrolidinyldicarboxyl groups. The carboxyl groups are bonded to amine-terminated R¹, R², R³ and R⁴ substituents, forming four (or three) amido linkages. The two pyrrolidinyldicarboxyl groups can also therefore also be referred to as two pyrrolidinyldicarboxamido groups.

Substituents bonded to the carboxyl groups of a pyrrolidinyldicarboxyl group can be in a cis or trans conformation, that is the two substituents can both project above or below the plane of the depicted ring (cis), or one can project above that plane and the other substituent project below (trans). A cis-disubstituted pyrrolidinyldicarboxyl group with two identical substituents has a symmetric configuration and does not have enantiomeric forms. A trans-disubstituted pyrrolidinyldicarboxyl group with those same two identical substituents has an asymmetric (chiral) configuration and has enantiomeric forms. The two chiral configurations are referred to as (S,S) and (R,R), and are shown below. It is

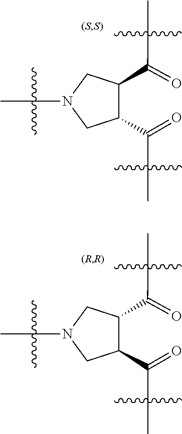

preferred that at least one, and more preferably both 3,4-pyrrolidinyldicarboxyl groups have the (S,S) configuration.

On the other hand, when -A of Formula V is hydrido and one of R¹⁻³ is a C₂-C₁₈ hydrocarbyl group, and more preferably, a C₁₀-C₁₆ hydrocarbyl group. A preferred compound corresponds in structure to Formula Va, below, in which the depicted R¹⁻³, W

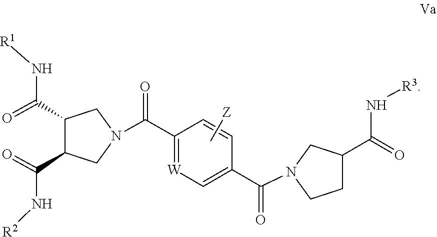

and Z moieties are as described above. The depicted —C(O)NH—R³ group can be in either the R configuration, the S configuration or present as a mixture of both configurations. The compounds with the S configuration are slightly more active and therefore more preferred.

In a compound of Formula Va, the number of carbon atoms of a R³ group here is preferably 2 to 18, and more preferably 10 to 16 carbon atoms. This hydrocarbyl group is also more preferably an alkyl group that is a straight chained substituent although methyl and ethyl branches can be tolerated as can double and/or triple bonds in the chain. Cyclic hydrocarbyl substituent compounds and carbocyclic ring-containing substituents can also be utilized.

It is also preferred that W be CH. Structural Formula Ia, shown below, incorporates several of the above preferences.

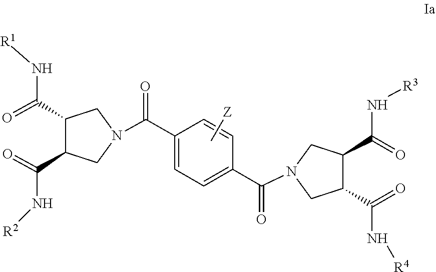

Structural formulas of currently most preferred compounds of Formula Ia in which —Z is hydrido (—H) are shown below as compounds of Formulas A, B, C, D, E, F, G, H, and I. Diprovocim numbers from WO 2018005812 are included for reference.

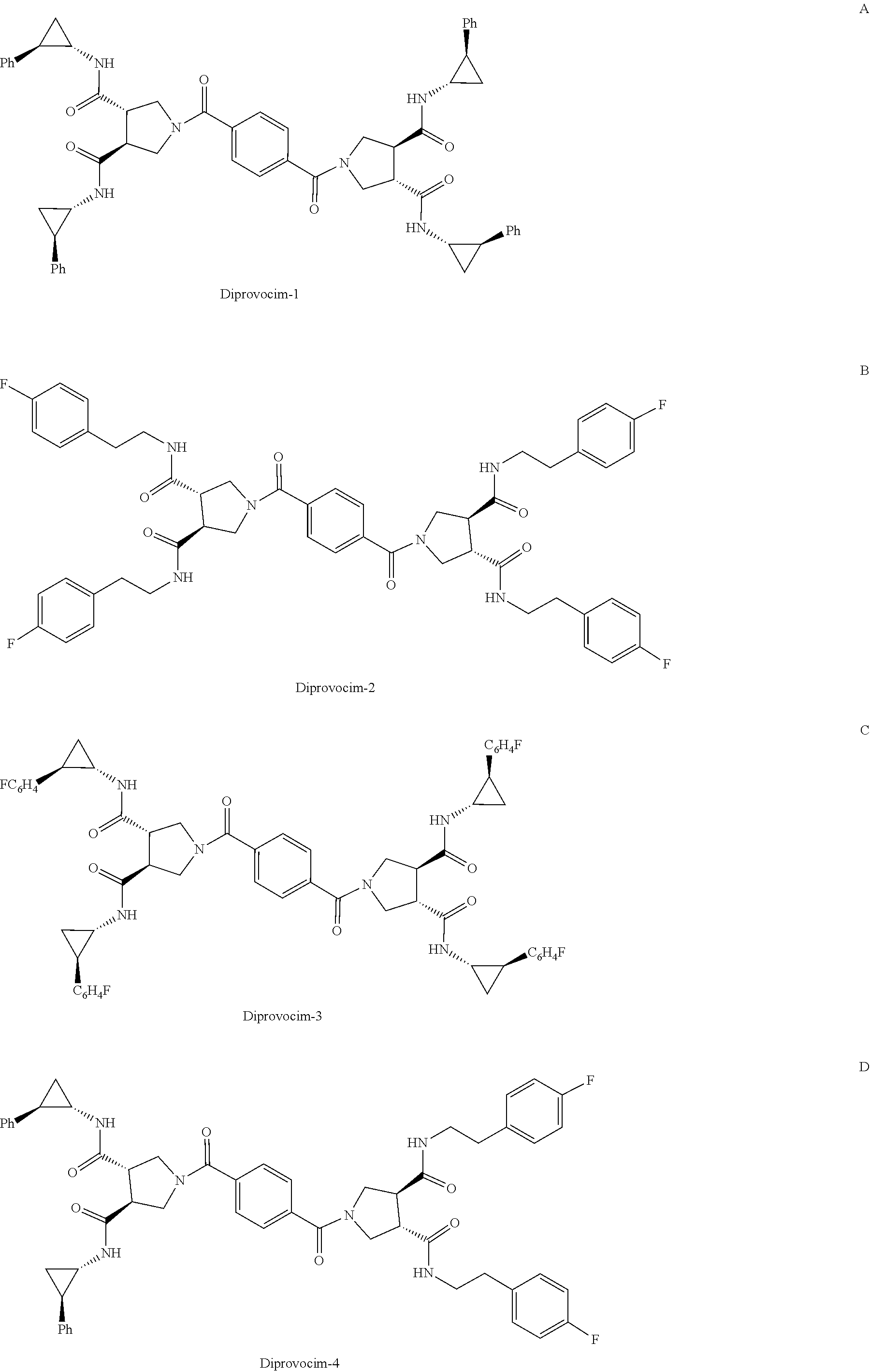

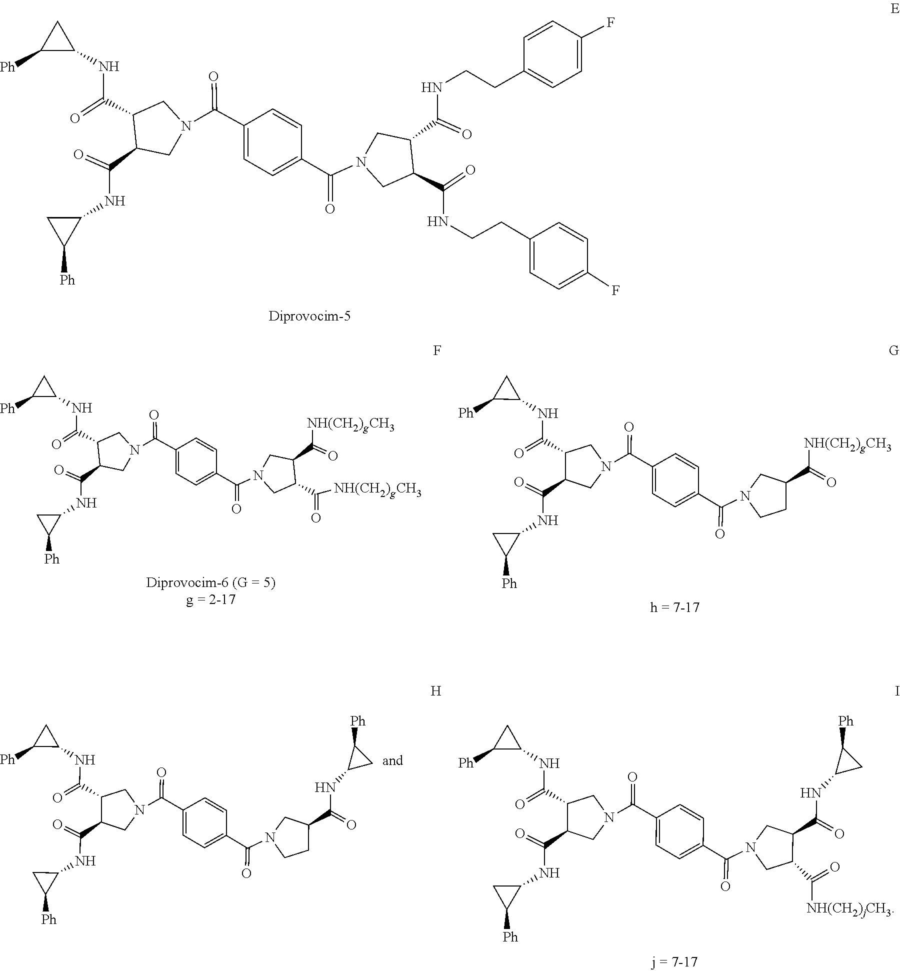

For a Compound of Formula F, “g” in both depicted amido groups is preferably the same number (length) of 5 to 9 methylene groups for maximal activity against both human THP-1 cells and mouse macrophages, with g=5 providing the most active compound for both cell types. For a Compound of Formula G, “h” in the depicted amido group is 1 to 17, preferably 7 to 17, and more preferably 9 to 15. For a Compound of Formula I, “j” in the depicted amido group is 1 to 17, preferably 3 to 11, and more preferably 5 to 9.

A contemplated immune checkpoint inhibitor is typically an intact antibody or the paratope-containing portion of an antibody. Such a contemplated antibody or antibody paratope-containing portion is preferably a monoclonal humanized, chimeric or human antibody. Illustrative checkpoint US FDA approved checkpoint inhibitors include Keytruda® (anti-PD-1), Yervoy® (anti-CTLA-4), Tecentriq® (anti-PD-L1), Opdivo® (anti-PD-1)], and Imfinzi® (anti-PD-L1)]. CTLA-4 itself binds to proteins B7-1 and B7-2 to inhibit T cell activity. Anti-B7-1 and anti-B7-2 paratope-containing molecules can also be used to block the CTLA-4/B7-1+B7-2 interaction, thereby providing checkpoint inhibitor activity much as do antibodies to either of the binding pair PD-1 and PD-L1.

Marker molecules are discussed in detail hereinafter. These materials are typically proteinaceous and can be the whole protein or an immunogenic portion of the protein.

When used in a method of inhibiting the growth of diseased cells in a mammal, an adjuvant-sufficient amount of a Diprovocim compound, a T cell-stimulating amount of an immune checkpoint inhibitor, and an immunizing amount of an antigenic (immunogenic) disease-related marker molecule are administered to contact host mammal cells. Those three components can be administered together, but it is preferred that the checkpoint inhibitor be administered separately from the Diprovocim and the antigen (immunogen) that can usually be administered together. Preferably, the checkpoint inhibitor is administered intravenously (IV), whereas the Diprovocim and antigen are administered together in an immunizing pharmaceutical composition intramuscularly (IM) or subcutaneously (SC).

Those administrations can be made within the span of a few minutes, hours, days or weeks. Many checkpoint inhibitors are antibodies or paratope-containing antibody portions that typically have an in vivo terminal half-life on the order of about 2 to about 4 weeks. [See, product label, Section 12.3 for Keytruda® (anti-PD-1), Yervoy® (anti-CTLA-4), Tecentriq® (anti-PD-L1), Opdivo® (anti-PD-1)], and Imfinzi® (anti-PD-L1)]. Thus, a checkpoint inhibitor can be administered prior to, coincidently with or a few days after administration of the Diprovocim and immunogen.

Each of the components can be administered a plurality of times during a course of treatment. The use of multiple administrations is illustrated herein.

A Diprovocim compound is administered in an adjuvant-sufficient amount. In the studies illustrated elsewhere herein, Diprovocim-1 (A) used illustratively herein acted as a robust in vivo adjuvant or TLR1/TLR2 agonist that evoked a potent TLR2-dependent adjuvant activity in vivo in mice at about 0.25 to about 5 mg/kg (i.m.) when co-injected with an immunogen in an immunizing pharmaceutical composition by an intramuscular route. An adjuvant-sufficient amount can be readily determined for mammals of greater weight by techniques well known in the art. In addition, Diprovocim-1 did not display the overt toxicity that is characteristic of LPS administration when used as an adjuvant.

A checkpoint inhibitor is typically utilized in an amount discussed in the product label. Illustrative dosage and administrations include the following using melanoma as exemplary diseased cell for use in the recited amounts: Keytruda®—melanoma: 2 mg/kg every 3 weeks; Yervoy®—adjuvant melanoma: 10 mg/kg administered intravenously over 90 minutes every 3 weeks for 4 doses, followed by 10 mg/kg every 12 weeks for up to 3 years or until documented disease recurrence or unacceptable toxicity; Tecentrie—administer 1200 mg as an intravenous infusion over 60 minutes every 3 weeks; and Opdivo—unresectable or metastatic melanoma 240 mg every 2 weeks.

An immunizing amount of an antigenic (immunogenic) disease-related marker molecule depends upon the immunogenicity of the marker used. The selection of peptides immunogenic for B cells and T cells is well known in the art and will not be gone into here. Many such useful peptides have been reported in the art but were not as successful formulated as vaccines as desired, presumably because of a lack of T cell help. It is believed that the three-part immunizing pharmaceutical composition overcomes that deficiency.

Many small disease-related marker molecules such as peptides having a length of about 5 to about 20 residues are themselves poorly immunogenic, and are often best utilized as haptens chemically linked to a carrier molecule. Illustrative proteinaceous carrier molecules include keyhole limpet hemocyanin (KLH), hepatitis B surface molecule (HBsAg), the hepatitis B core (capsid; HBcAg), ovalbumin, bovine serum albumin, bovine gammaglobulin and human gammaglobulin have been used as a hapten carrier, as have many other molecules have been used in the literature.

A contemplated disease-related marker molecule is present in and/or on diseased cells. Diseased cells are typically cancerous or pathogen-infected.

Disease-related marker molecules or portions thereof useful as immunogens present in and/or on solid tumor cells, include cancer stem cell (CSC) markers that are rarely expressed on normal tissue cells are listed below. [Kim et al., BMB Rep 50(6):285-298 (2017).]. Exemplary CSC markers that are largely absent in normal (disease-free) cells and present in diseased cells include CD96, CD20, DLL4, CD55, TIM-3, CXCR1, CD54, CD114, LGR5, CD105, CD56, CD13, CD271, CD34, CXCR4, CD26, CD117, CD10, CD146, Notch2, CD49f, CD24, ABCG2, PODXL-2, Cripto-1, CD326, CD90, CD133, SSEA1, TRA-1-81, TRA-1-60, SSEA4, SSEA3, CD151, CD340 and CD44.

Exemplary disease-related marker molecules are typically present in and/or on solid tumor cells. Illustrative solid tumors include osteosarcoma cells, Kaposi's sarcoma cells, melanoma cells, prostate cancer cells, glioblastoma cells, small cell lung carcinoma cells, breast cancer cells, liver cancer cells, colon cancer cells, ovarian cancer cells, renal cancer cells, gastric cancer cells, neuroblastoma cells, pancreatic cancer cells, and Hodgkin's lymphoma cells.

A contemplated disease-related marker molecule or portion thereof can also be present in and/or on pathogen-infected cells. Illustrative infecting pathogens include one or more of a virus, bacterium, fungus and unicellular parasite.

Illustrative viruses include influenza, hepatitis viruses A, B, C and D, herpes viruses such as Varicella zoster (chickenpox), Herpes simplex 1 and 2 (HSV1 and HSV2), human papilloma virus (HPV), and the like. Illustrative bacterial pathogens include E. coli, E. faecalis, S. aureus, and the like. An illustrative unicellular parasite is the malaria sporozoite of P. falciparum, P. vivax, P. bergeii or P. yoelli.

Illustrative proteinaceous immunogens include the following disease-related marker molecule peptides that are listed below with a citation to their publication source.

U.S. Pat. No. 6,942,866

Malarial B Cell Epitopes

| | |
|---|---|
| P. falciparum | |
| (NANP)₄ | SEQ ID NO: |
| NANPNVDP(NANP)₃NVDP | SEQ ID NO: |
| NANPNVDP(NANP)₃ | SEQ ID NO: |
| (NANP)₃NVDPNANP | SEQ ID NO: |
| NANPNVDP(NANP)₃NVDPNANP | SEQ ID NO: |
| NPNVDP(NANP)₃NV | SEQ ID NO: |
| NPNVDP(NANP)₃NVDP | SEQ ID NO: |
| NPNVDP(NANP)₃NVDPNA | SEQ ID NO: |
| NVDP(NANP)₃NV | SEQ ID NO: |
| NVDP(NANP)₃NVDP | SEQ ID NO: |
| NVDP(NANP)₃NVDPNA | SEQ ID NO: |
| DP(NANP)₃NV | SEQ ID NO: |
| DP(NANP)₃NVDP | SEQ ID NO: |
| DP(NANP)₃NVDPNA | SEQ ID NO: |
| P. vivax | |
| DRAAGQPAGDRADGQPAG | SEQ ID NO: |
| ANGAGNQPGANGAGDQPGA- | SEQ ID NO: |
| NGADNQPGANGADDQPG | |
| ANGAGNQPGANGAGDQPG | SEQ ID NO: |
| ANGADNQPGANGADDQPG | SEQ ID NO: |
| ANGAGNQPGANGADNQPG | SEQ ID NO: |
| ANGADNQPGANGADDQPG | SEQ ID NO: |
| APGANQEGGAAAPGANQEGGAA | SEQ ID NO: |
| P. bergeii | |
| (DP₄NPN)₂ | SEQ ID NO: |
| P. yoelli | |
| (QGPGAP)₄ | SEQ ID NO: |

Malarial Universal T Cell Epitope

| | |
|---|---|
| P. falciparum | |
| GIEYLNKIQNSLSTEWSPCSVT | SEQ ID NO: |
| P. vivax | |
| YLDKVRATVGTEWTPCSVT | SEQ ID NO: |
| P. yoelli | |
| EFVKQISSQLTEEWSQCSVT | SEQ ID NO: |

U.S. Pat. No. 8,017,127

Influenza A M2 Protein B Cell Epitopes

As is noted in U.S. Pat. No. 8,017,127, the M2 protein is expressed in cells infected by the influenza A strains. The N-terminal residues 1-24 of the M2 protein extends through the infected cell's membrane. That extracellular portion of the protein is referred to as M2e. As a consequence, use of the influenza A extracellular M2e portion of that protein as the immunogenic marker can provide protection from all of the influenza strains. Thus, the yearly changes in influenza vaccine selection can be avoided.

| Sequence | SEQ ID NO |
|---|---|
| SLLTEVETPIRNEWGCRCNGSSD | |
| SLLTEVETPIRNEWGCRCNDSSD | |
| SLLTEVETPIRNEWGARANDSSD | |
| SLLTEVETPIRNEWGSRSNDSSD | |
| SLLTEVETPIRNEWGSRCNDSSD | |
| SLLTEVETPIRNEWGCRSNDSSD | |
| SLLTEVETPIRNEWGCRANDSSD | |
| SLLTEVETPIRNEWGARCNDSSD | |
| MSLLTEVETPIRNEWGCRCNDSSD | |
| MSLLTEVETPIRNEWGSRSNDSSD | |
| MGISLLTEVETPIRNEWGCRCND- | |
| SSDELLGWLWGI | |
| MSLLTEVETPIRNEWGARANDSSD | |
| MSLLTEVETPIRNEWGCRANDSSD | |
| MSLLTEVETPIRNEWGARCNDSSD | |
| MSLLTEVETPIRNEWGCRSNDSSD | |
| MSLLTEVETPIRNEWGSRCNDSSD | |
| SLLTEVETPIRNEWGSRSNDSSDSLL- | |
| TEVETPIRNEWGSRSNDSSD | |
| SLLTEVETPIRNEWGSRSNDSSDSLL- | |
| TEVETPIRNEWGSRSNDSSDSLL- | |
| TEVETPIRNEWGCRCNDSSD | |
| SLLTEVETPIRNEWGARANDSSDSLL- | |
| TEVETPIRNEWGCRCNDSSD | |
| SLLTEVETPIRNEWGARANDSSDSLL- | |
| TEVETPIRNEWGARANDSSDSLL- | |
| TEVETPIRNEWGCRCNDSSD | |
| EVETPIRNEWGSRCNDSSD | |
| EVETPIRNEWGSRCNDSSDEVET- | |
| PIRNEWGSRCNDSSD | |
| EVETPIRNEWGSRCNDSSDEVET- | |
| PIRNEWGSRCNDSSDEVE- | |
| TPIRNEWGCRCNDSSD | |
| SLLTEVETPIRNEWGSRSNDSSDSLL- | |
| TEVETPIRNEWGSRSNDSSDSLL- | |
| TEVETPIRNEWGSRSNDSSDSLL- | |
| TEVETPIRNEWGCRCNDSSD | |
| SLLTEVETPIRNEWGSRSNDSSDSLL- | |
| TEVETPIRNEWGSRSNDSSDSLL- | |
| TEVETPIRNEWGSRSNDSSDSLL- | |
| TEVETPIRNEWGSRSNDSSDSLL- | |
| TEVETPIRNEWGCRCNDSSD | |
| SLLTEVETPTRNEWGCRCNDSSD | |
| SLLTEVETPTRNGWGCRCNDSSD | |
| SLLTEVETPIRNEWECRCNGSSD | |
| SLLTEVETPTKNEWECRCNDSSD | |
| SLLTEVETPTRNGWECKCSDSSD | |
| SLLTEVDTLTRNGWGCRCSDSSD | |
| SLLTEVETLTRNGWECKCRDSSD | |
| Influenza B Protein | |
| NNATFNYTNVNPISHIR | |

U.S. Pat. No. 4,599,231

Hepatitis B Virus Surface Antigen

The hepatitis B virus surface antigen (HBsAg) provides both B cell and T cell polypeptide epitopes. A number of each epitope type as disclosed in U.S. Pat. No. 4,599,231 are set out below in the table along with their peptide denominations, and parenthesized sequence position from the N-terminus, as recited in that patent based on DNA from an ayw donor (P49) and an adw donor (P72 and P73).

| Sequence | SEQ ID NO |
|---|---|
| B Cell Epitope | |
| P49 (110-137) | |
| FPGSSTTSTGPCRTCMTTAQGTSMYPSC | |
| P49a (125-137) | |
| MTTAQGTSMYPSC | |
| P72 (125-137) | |
| IPGSTTTSTGPCKTCTTPAQGNSMFPSC | |
| P72a (125-137) | |
| TTPAQGNSMFPSC | |
| P73 (107-137) | |
| CPLIPGSTTTSTGPCKTCTTPAQGNSMFPSC | |
| T Cell Epitope | |
| P1 (48-81) | |
| CLGQNSQSPTSNHSPTSCPPTCPGYRWMCLRRF I | |
| P5 (38-52) | |
| SLNFLGGTTVCLGQN | |
| P5a (74-52) | |
| VCLGQN | |
| P6 (95-109) | |
| LIFLLVLLDYQGMLPVCPL | |

U.S. Pat. No. 5,180,806

Human Papilloma Virus (HPV) Marker Peptides

Papillomaviruses induce benign, dysplastic and malignant hyperproliferations of skin or mucosal epithelium. More than 50 types (strains) of human papillomavirus (HPV) have been identified. In humans, different papillomavirus types are known to cause distinct diseases. For example, HPV types 1 and 2 cause common warts, and types 6 and 11 cause condylomas and genital flat warts. In contrast, HPV types 16, 18 and 33 are carried in a majority of cervical cancers and do not cause the usual condyloma, but rather persist diffusely in the cervical endothelium exhibiting only minimal pathologic changes. It is thought that the HPV types associated with cervical cancer are maintained in a latent state in cervical endothelium tissues for years after initial infection and then progress in some cases to cause cervical cancer.

U.S. Pat. No. 5,180,806 discloses several peptide sequences that induce the production of antibodies. Illustrative peptide markers of type 16-related HPV sequences disclosed in U.S. Pat. No. 5,180,806 are set out below as illustrative. That patent also discloses peptide sequences from type 18 and type 33, as well as sequences encoded by the E2 ORF of HPV types 6, 11, 18 and 33.

HPV Type 16-Related Polypeptides

| Sequence | SEQ ID NO |
|---|---|
| MADPAGTNGEEGTGC | |
| HEDEDKENDGDSLPTC | |
| RPFKSNKSTCC | |
| CCDWCIAAFGLTPSI | |
| TYDSEWQRDQFLSQVKIPC | |
| HKSAIVTLTYDSEWQRDQC | |
| CINCQKPLCPEEKQRH | |

A contemplated immunizing composition also typically contains pharmaceutically acceptable salts, buffers and the like excipients that collectively are referred to as pharmaceutically (or physiologically) acceptable diluents or carriers as compared to those that can be present in a composition that is not intended for pharmaceutical use, as in an in vitro assay. These compositions are discussed in further detail hereinafter.

A Diprovocim compound useful herein can be provided for use by itself, or as a pharmaceutically acceptable salt. Exemplary salts useful for a contemplated compound include but are not limited to the following: sulfate, hydrochloride, hydro bromides, acetate, adipate, alginate, citrate, aspartate, benzoate, benzenesulfonate, bisulfate, butyrate, camphorate, camphorsulfonate, digluconate, cyclopentanepropionate, dodecylsulfate, ethanesulfonate, glucoheptanoate, glycerophosphate, hemisulfate, heptanoate, hexanoate, fumarate, hydrochloride, hydrobromide, hydroiodide, 2-hydroxy-ethanesulfonate, lactate, maleate, methanesulfonate, nicotinate, 2-naphthalenesulfonate, oxalate, palmoate, pectinate, persulfate, 3-phenyl-propionate, picrate, pivalate, propionate, succinate, tartrate, thiocyanate, tosylate, mesylate and undecanoate. Salts of the carboxylate group include sodium, potassium, magnesium, calcium, aluminum, ammonium, and the many substituted ammonium salts.

The reader is directed to Berge, J. Pharm. Sci. 1977 68(1):1-19 for lists of commonly used pharmaceutically acceptable acids and bases that form pharmaceutically acceptable salts with pharmaceutical compounds.

In some cases, the salts can also be used as an aid in the isolation, purification or resolution of the compounds of this invention. In such uses, the acid or base used and the salt prepared need not be pharmaceutically acceptable.

A contemplated immunizing pharmaceutical composition contains an adjuvant-effective amount of a Compound of Formula V or a pharmaceutically acceptable salt thereof dissolved or dispersed in a physiologically (pharmaceutically) acceptable carrier along with the immunogenic marker. Such a composition can be administered to mammalian cells in vitro as in a cell culture to contact those cells, or the cells can be contacted in vivo as in a living, host mammal in need. As is seen from the data herein, a contemplated Diprovocim compound present at femtomolar to nanomolar amounts provides an adjuvant effect in in vivo and in in vitro assay studies.

When used as a vaccine adjuvant, a Compound of Formula V is preferably administered together with the selected marker immunogen. Both components are preferably present together in a single immunizing pharmaceutical composition as noted above. However, the two ingredients can be present in separately administered immunizing pharmaceutical compositions, and those separate compositions can be administered up to about one to about two hours apart. It is preferred when two separate compositions are administered, that they be administered as close together in time as possible.

In another embodiment, some or all of the Diprovocim compound utilized can be chemically bonded to the immunizing marker compound. That chemical bond can be formed using the Z substituent shown in Formula V as where a Z substituent that includes a carboxyl group can be bonded to an amino group of an immunogenic peptide marker compound. Alternatively, where an immunogenic marker compound is utilized as a hapten bonded to a carrier molecule, the Diprovocim compound can also be chemically bonded to the same carrier molecule.

A contemplated immunizing pharmaceutical composition is typically administered in vivo to a subject in need thereof a plurality of times within one month, such as daily or weekly, and can be administered over a period of several months to several years. More usually, a contemplated composition is administered a plurality of times over a course of treatment.

A contemplated immunizing pharmaceutical composition is preferably adapted for parenteral administration. Thus, an immunizing pharmaceutical composition is preferably in liquid form when administered, and most preferably, the liquid is an aqueous liquid, although other liquids are contemplated as discussed below, and a presently most preferred composition is an injectable preparation.

Thus, injectable preparations, for example, sterile injectable aqueous or oleaginous solutions or suspensions can be formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be employed are water, Ringer's solution, and isotonic sodium chloride solution, phosphate-buffered saline.

Other liquid pharmaceutical compositions include, for example, solutions suitable for parenteral administration. Sterile water solutions of a Compound of Formula V or sterile solution of a Compound of Formula V in solvents comprising water, ethanol, or propylene glycol are examples of liquid compositions suitable for parenteral administration. In some aspects, a contemplated Compound of Formula V is provided as a dry powder that is to be dissolved in an appropriate liquid medium such as sodium chloride for injection prior to use.

In addition, sterile, fixed oils are conventionally employed as a solvent or suspending medium. For this purpose any bland fixed oil can be employed including synthetic mono- or diglycerides. In addition, fatty acids such as oleic acid find use in the preparation of an injectable composition. Dimethyl acetamide, surfactants including ionic and non-ionic detergents, polyethylene glycols can be used. Mixtures of solvents and wetting agents such as those discussed above are also useful. Sterile solutions can be prepared by dissolving the active component in the desired solvent system, and then passing the resulting solution through a membrane filter to sterilize it or, alternatively, by dissolving the sterile compound in a previously sterilized solvent under sterile conditions.

A mammal in having diseased cells in need of treatment (a subject) and to which a pharmaceutical composition containing at least a Compound of Formula V and a immunogenic marker compound is administered can be a primate such as a human, an ape such as a chimpanzee or gorilla, a monkey such as a cynomolgus monkey or a macaque, a laboratory animal such as a rat, mouse or rabbit, a companion animal such as a dog, cat, horse, or a food animal such as a cow or steer, sheep, lamb, pig, goat, llama or the like.

Preferably, the pharmaceutical composition is in unit dosage form. In such form, the composition is divided into unit doses containing appropriate quantities of the Diprovocim and immunogen. The unit dosage form can be a packaged preparation, the package containing discrete quantities of the preparation, for example, in vials or ampules.

Results

Diprovocim Induces Cytokine Production in Both Human and Mouse Cells

From a chemical library containing approximately 100,000 members, a class of compounds was identified with bilateral symmetry capable of activating TNF biosynthesis in PMA-differentiated human THP-1 myeloid cells. The initial members of the class emerged from an undisclosed compound sublibrary designed to promote cell surface receptor dimerization (18).

Diprovocim-1, below, was developed from this class after extensive structure-activity

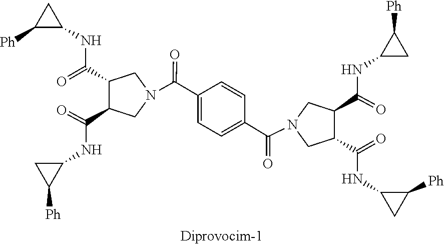 [SMILES: O=C(N[C@H]1C[C@@H]1c1ccccc1)[C@@H]1CN(C(=O)c2ccc(C(=O)N3C[C@@H](C(=O)N[C@H]4C[C@@H]4c4ccccc4)[C@H](C(=O)N[C@H]4C[C@@H]4c4ccccc4)C3)cc2)C[C@H]1C(=O)N[C@H]1C[C@@H]1c1ccccc1]

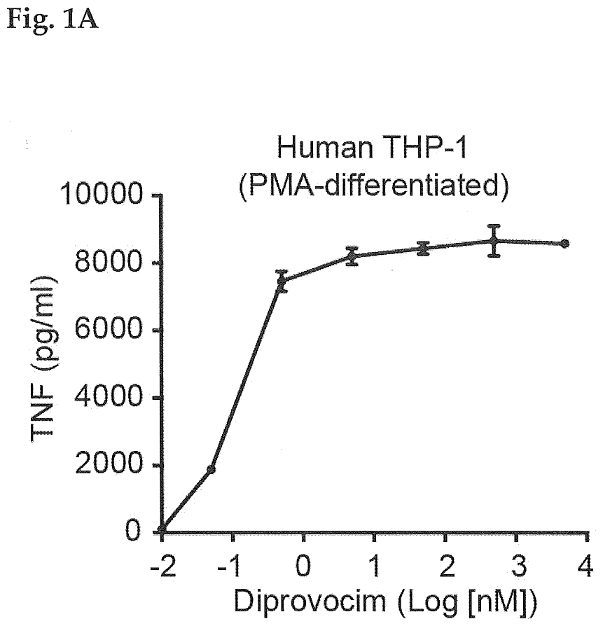

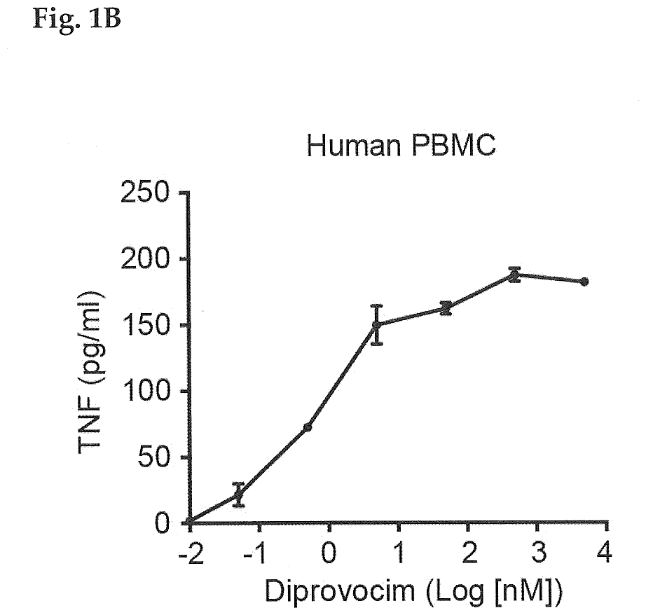

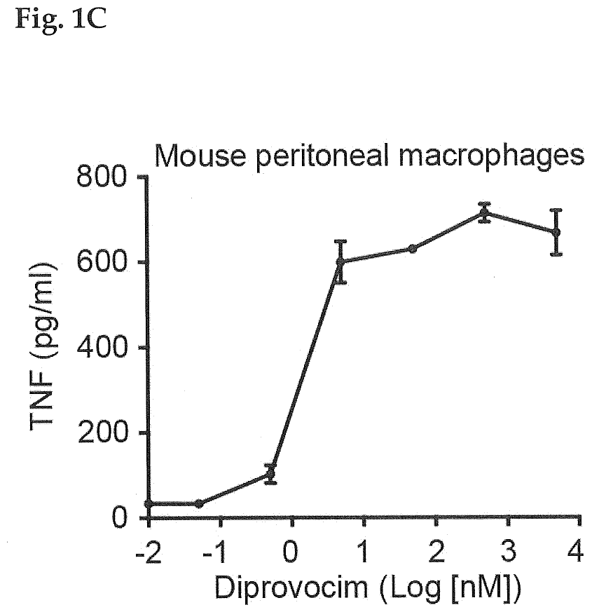

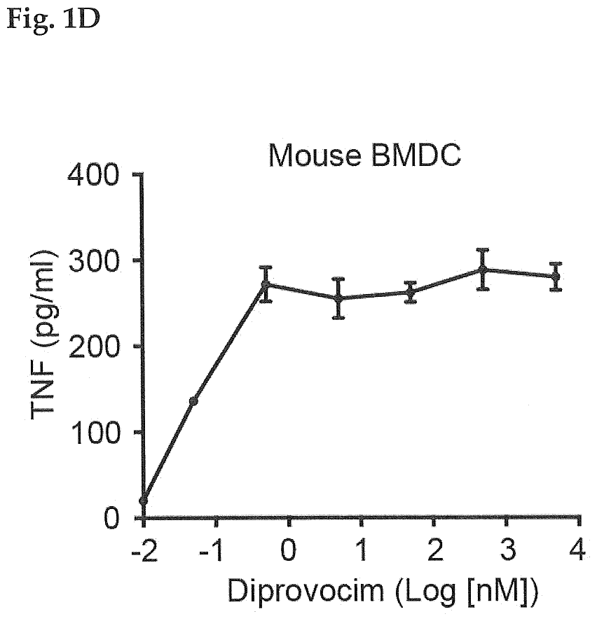

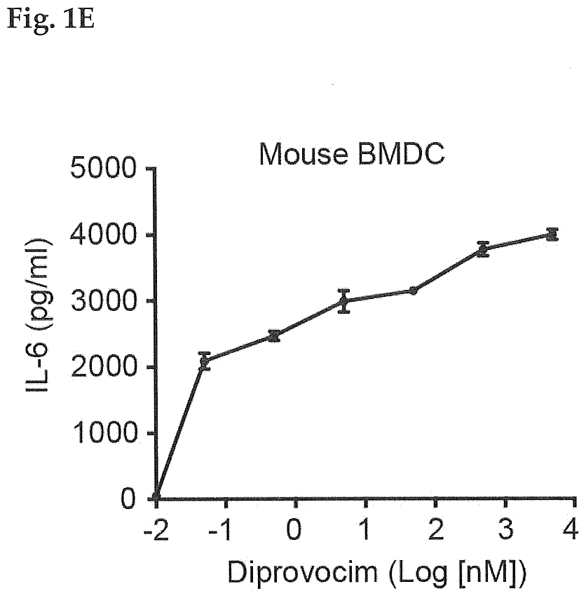

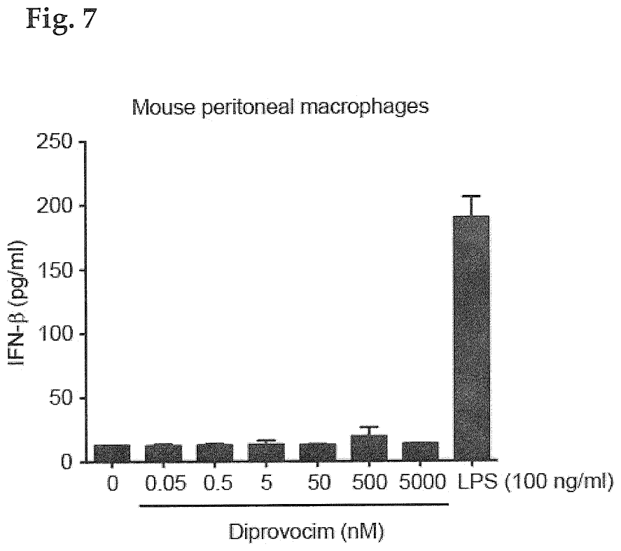

relationship (SAR) studies. It induced dose-dependent TNF production by THP-1 cells (EC₅₀ 110 pM) and human PBMC (EC₅₀ 875 pM) (FIG. 0A and FIG. 1B), and by mouse peritoneal macrophages (EC₅₀ 1.3 nM) and bone marrow-derived dendritic cells (BMDC) (EC₅₀ 6.7 nM) (FIG. 0C and FIG. 1D). In addition to TNF, Diprovocim-1 induced IL-6 production by mouse BMDC (FIG. 0E). However, Diprovocim-1 failed to stimulate type I IFN production by mouse peritoneal macrophages (FIG. 7). The other numbered Diprovocims studied and those defined by structural Formula I also share these activities.

Diprovocim Targets TLR1/TLR2 and Activates Downstream MAPK and NF-κB Signaling Pathway

To determine the molecular target of the Diprovocims, effects were analyzed on peritoneal macrophages from wild type C57BL/6J mice and C57BL/6J mice deficient in various TLR signaling components. Induction of TNF by Diprovocim was completely absent in TLR1- or TLR2-deficient macrophages, but not in TLR-6 deficient macrophages (FIG. 1A). Diprovocim activity was also dramatically reduced in macrophages from MyD88-, TIRAP-, and IRAK4-deficient cells (FIG. 1A). These data suggested that Diprovocim targets the mouse TLR1/TLR2 heterodimer.

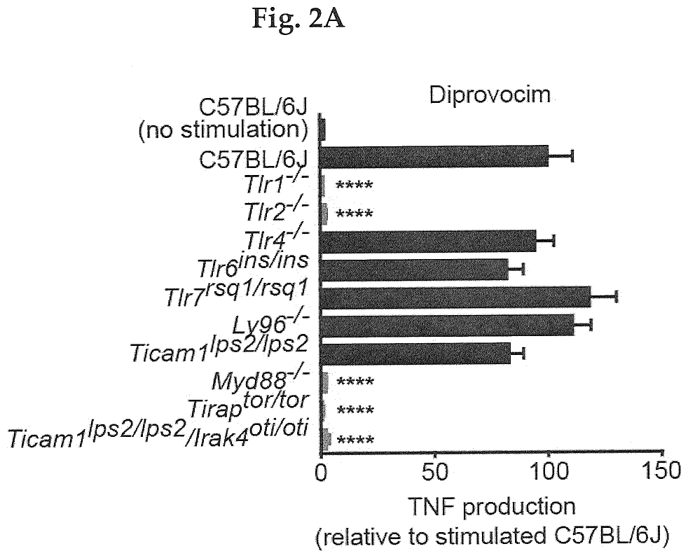

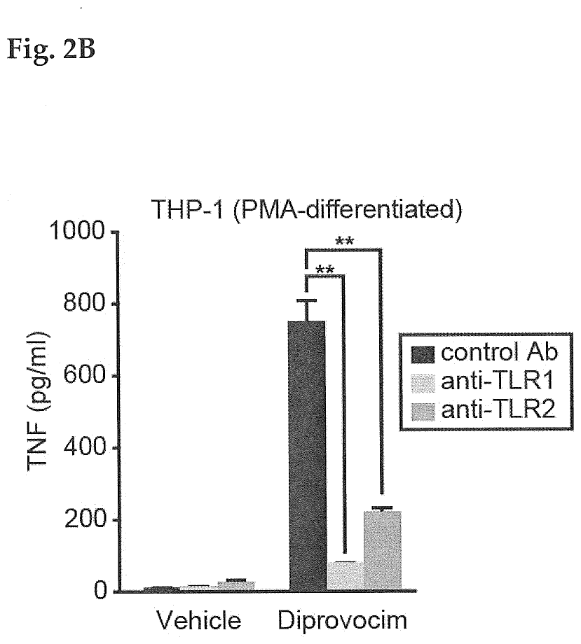

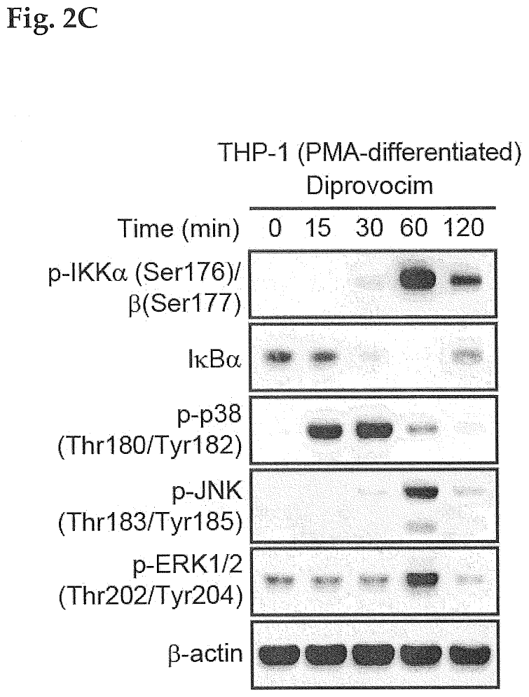

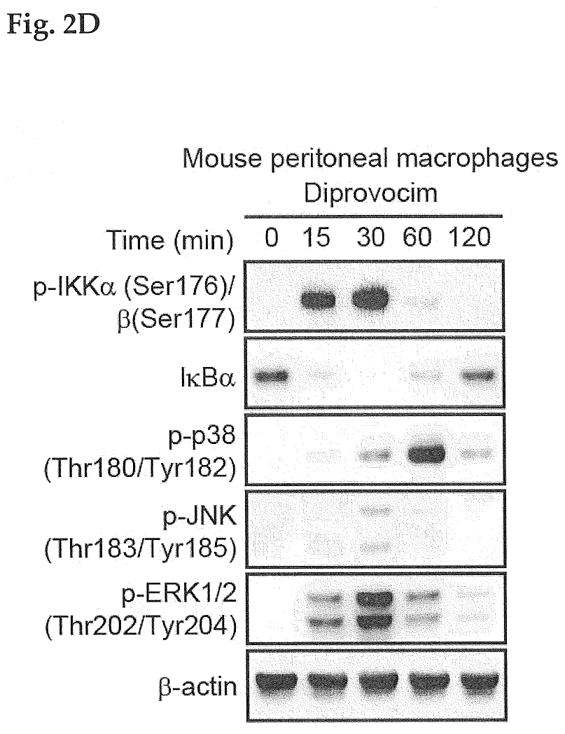

TLR1 or TLR2 antibody significantly reduced the effect of Diprovocim on THP-1 cells, indicating that human TLR1/TLR2 is also a target of Diprovocim molecules (FIG. 1B). Diprovocim induced phosphorylation of IκKα, IKKβ, p38, JNK, and ERK, as well as degradation of IκBα in THP-1 cells and mouse peritoneal macrophages, indicating that Diprovocims activate conventional TLR1/TLR2 signaling, including MAPK and canonical NF-κB signaling (FIG. 1C and FIG. 2D).

Diprovocim Exhibits Adjuvant Activity In Vivo

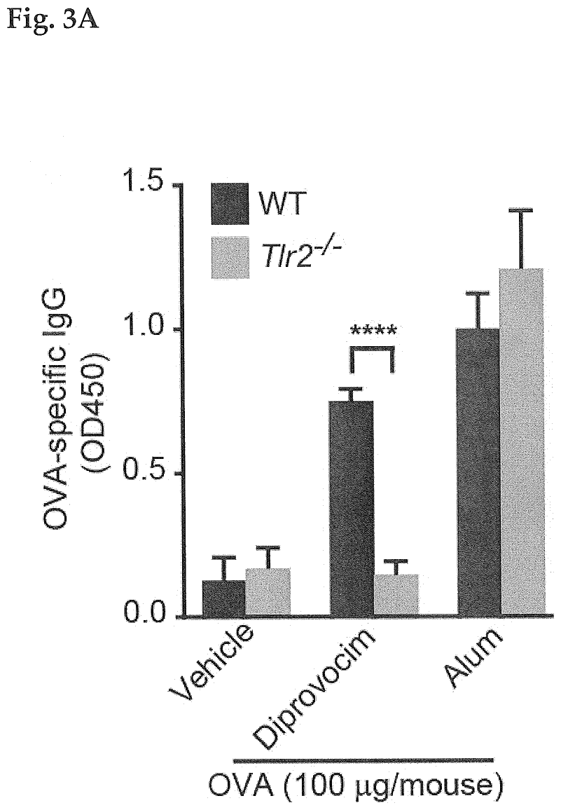

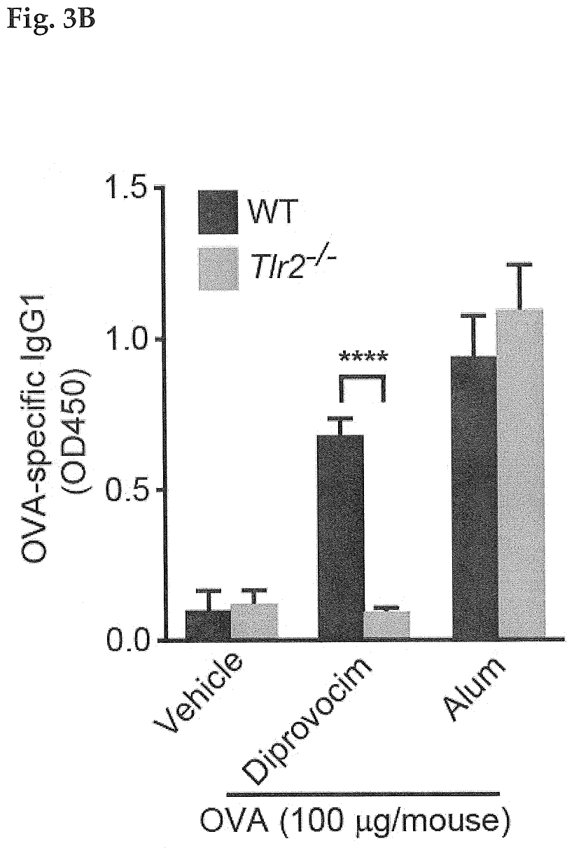

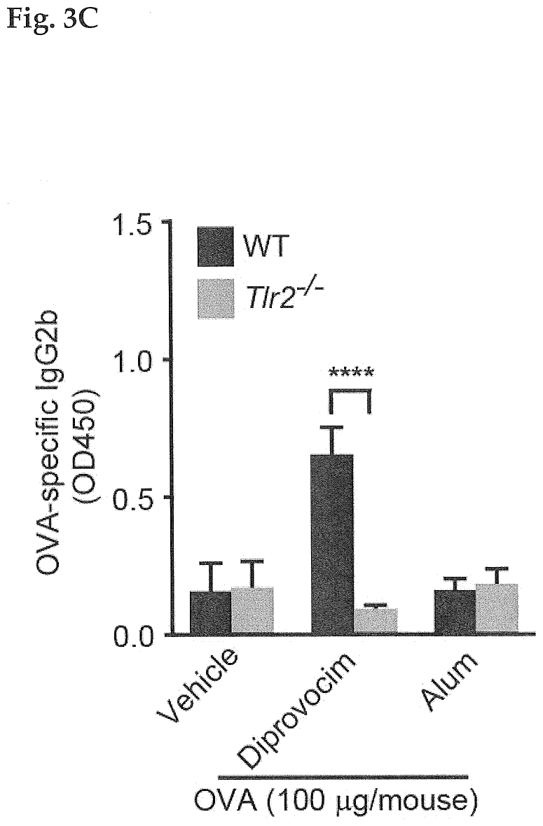

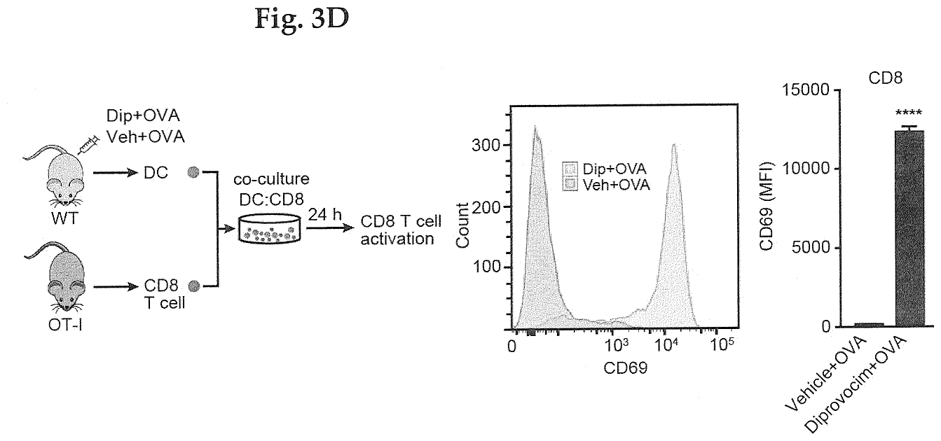

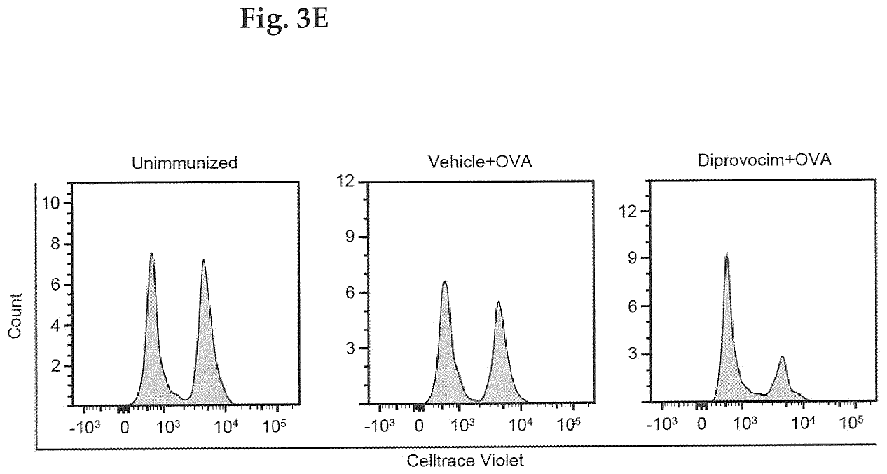

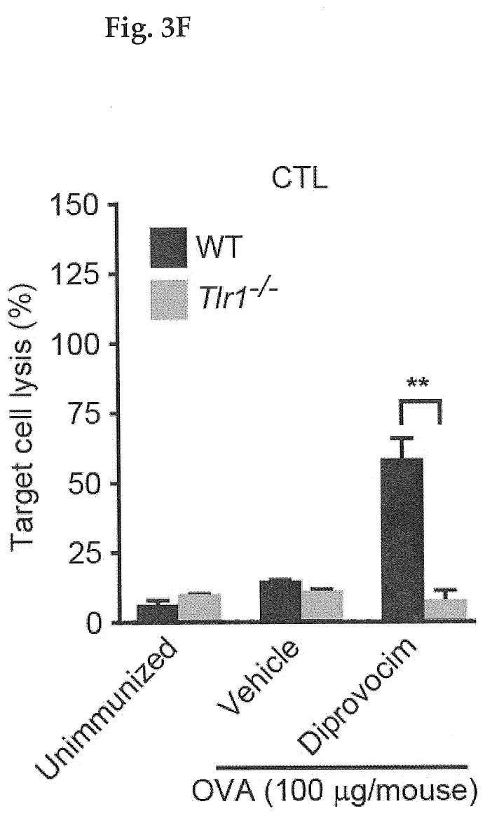

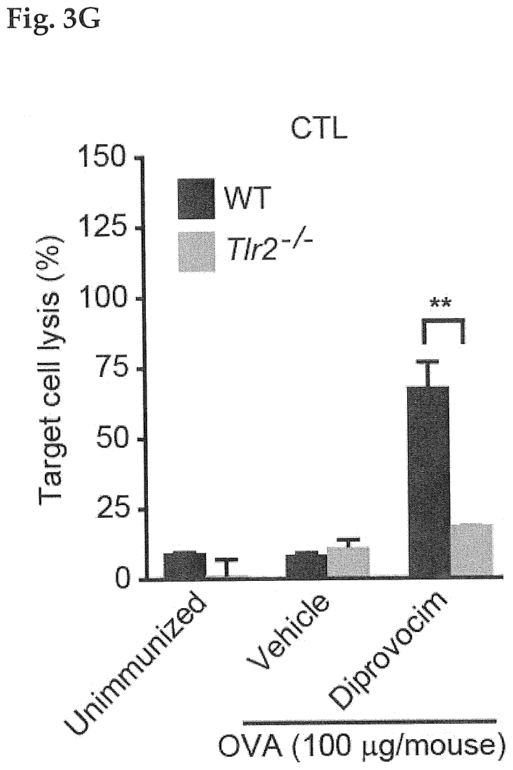

Intramuscular immunization of wild type mice with ovalbumin (OVA) plus either alum or Diprovocim induced similar levels of serum OVA-specific IgG, which were highly elevated compared to levels induced by immunization with OVA plus vehicle (FIG. 2A-FIG. 3C). Whereas immunization with OVA+alum induced primarily the Th2-related Ig subclass IgG1, OVA+Diprovocim induced both IgG1 and the Th1-related IgG2b (FIG. 2B and FIG. 3C).

Dendritic cells (DCs) purified from draining lymph nodes and spleens 24 hours after immunization of mice with OVA+Diprovocim activated OT-I CD8 T cells co-cultured with them, as evidenced by CD69 upregulation on the OT-I cells (FIG. 2D). In contrast, DCs from mice immunized with OVA+vehicle failed to induce CD69 expression on OT-I CD8 T cells (FIG. 2D). This finding suggests that Diprovocims activate antigen cross-presentation by DCs and cross-priming of CD8 T cells in vivo.

To further investigate whether cross-priming stimulated by a Diprovocim results in the development of killing ability by CD8+ T cells in vivo, a CTL killing assay was performed. Fluorescent marker-labeled and OVA peptide (residues, 257-263)-pulsed target cells were injected i.v. into mice immunized with OVA+Diprovocim and the number of live target cells was measured by flow cytometry two days later. About 70% of target cells had been eliminated in mice immunized with OVA+Diprovocim, compared to about 10% in mice immunized with OVA+vehicle (FIG. 2E). These data demonstrate that Diprovocim exhibits adjuvant activity in antigen-specific antibody production and CTL killing, which was abrogated in TLR1- and/or TLR2-deficient mice (FIGS. 2 A-C, F and G).

Complete Inhibition of B16 Tumor Growth by Combined Checkpoint Blockade and Anti-Cancer Vaccine Adjuvanted with Diprovocim

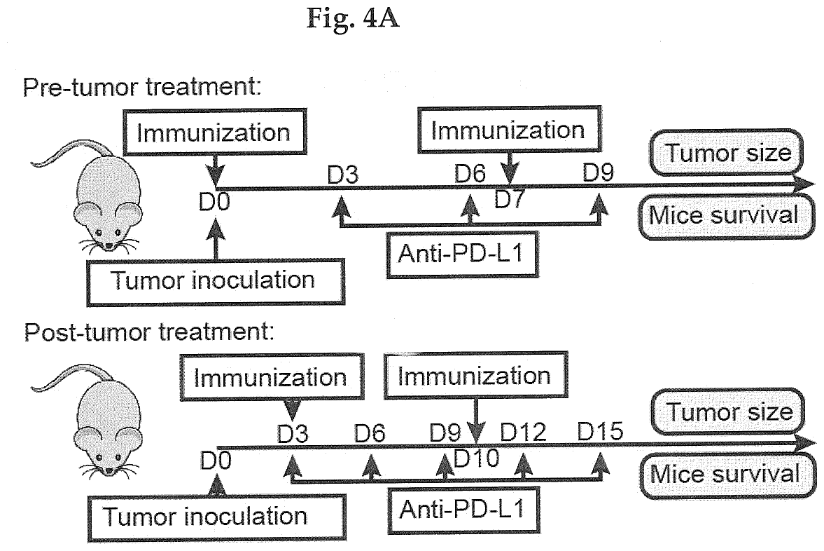

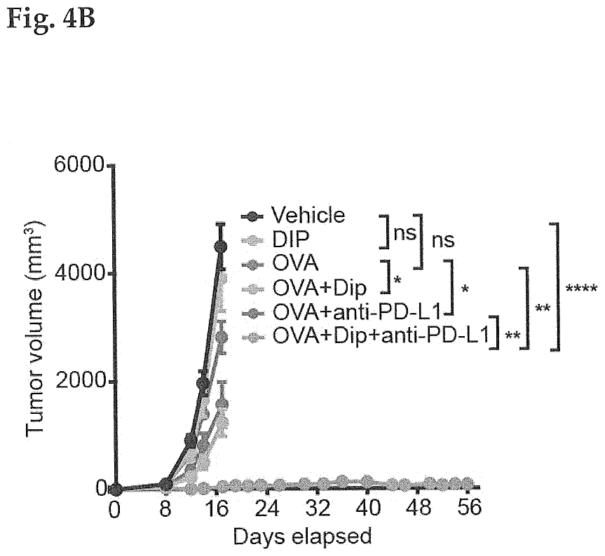

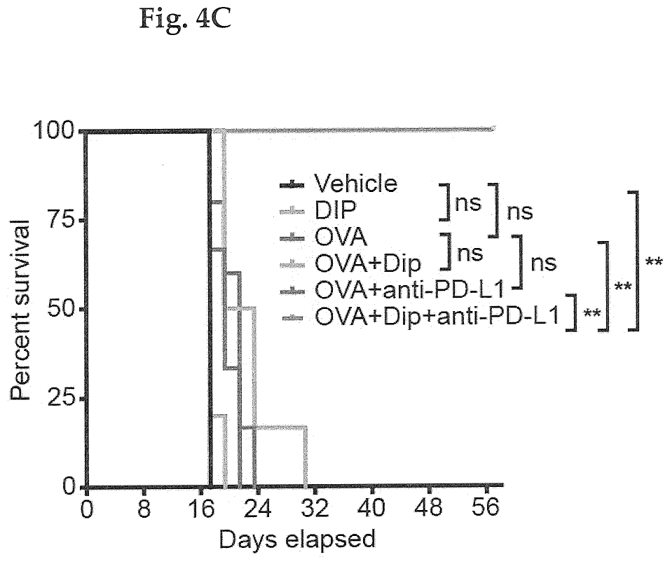

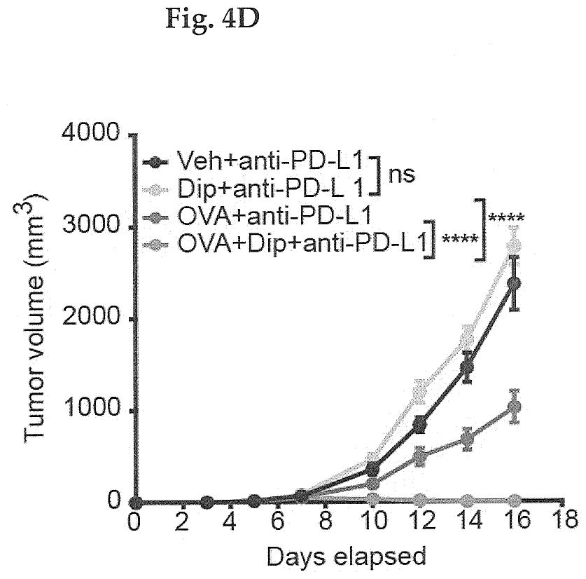

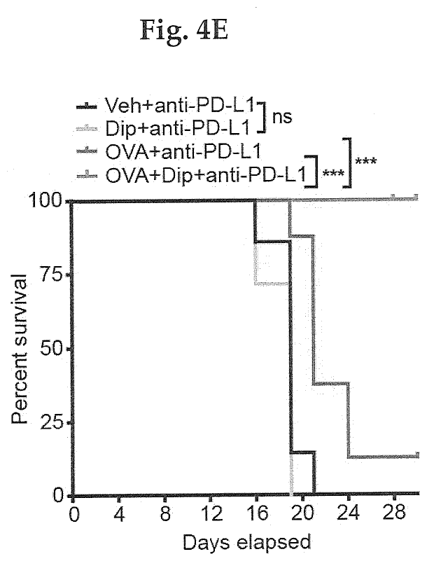

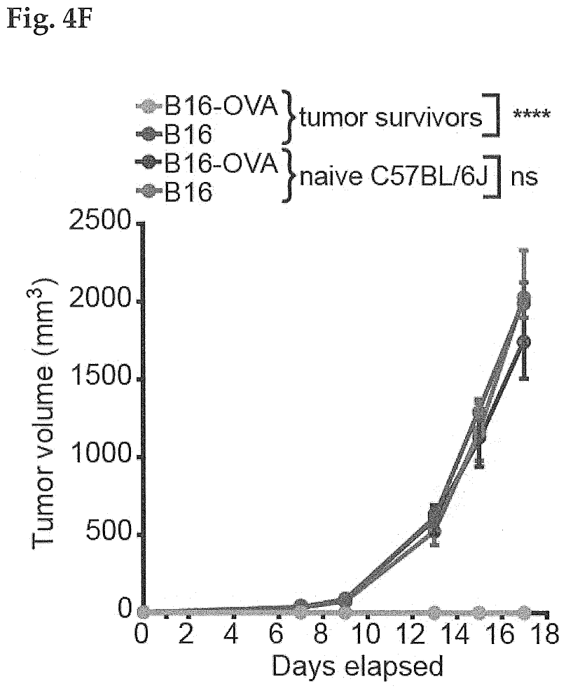

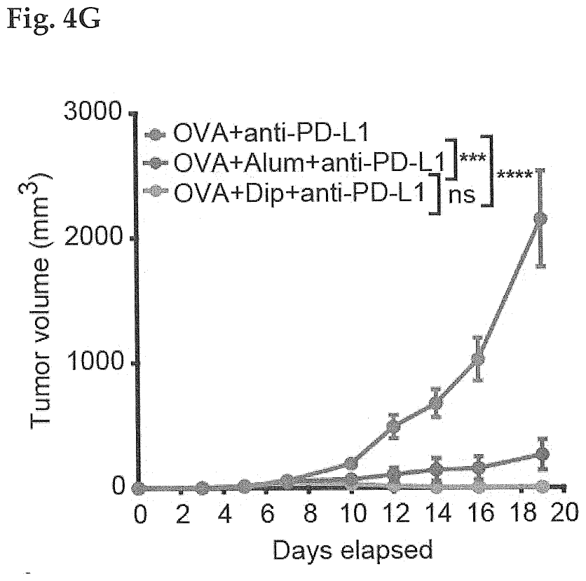

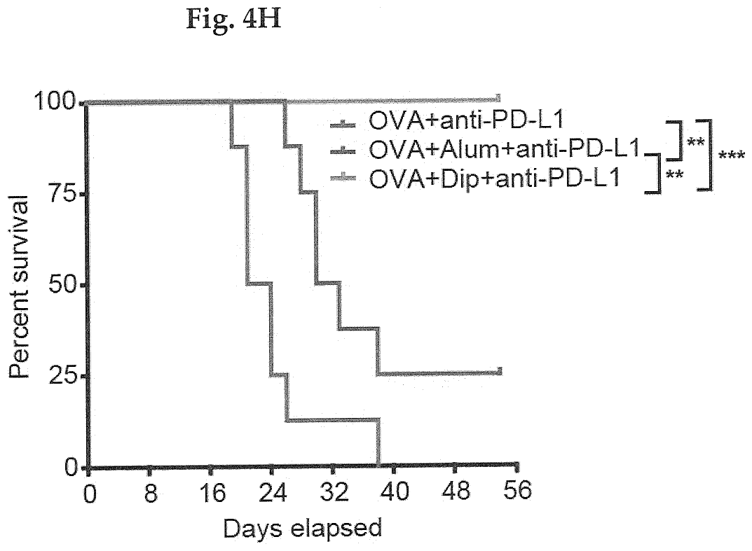

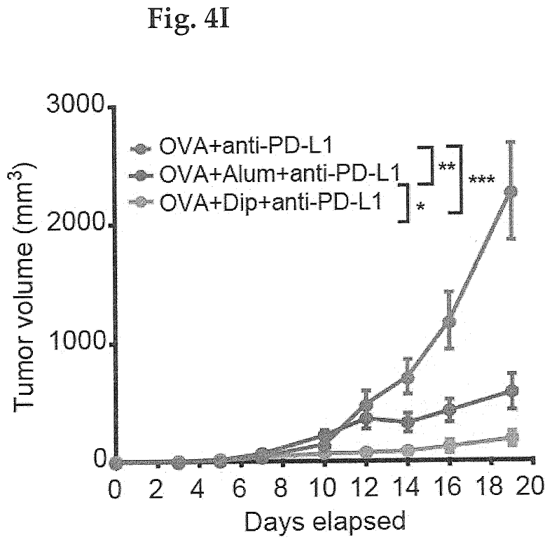

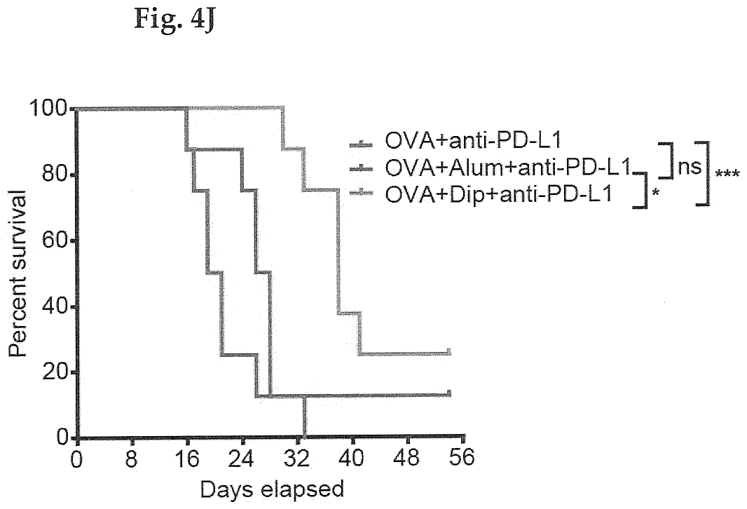

The adjuvant activity of Diprovocim was examined in preventive immunization of wild type mice against B16 melanoma expressing OVA (B16-OVA) (FIG. 3A). Mice were injected i.m. distal to the tumor cell injection site with OVA with or without Diprovocim on the same day but prior to inoculation with B16-OVA cells. Tumor growth rates and survival times were similar for mice immunized with vehicle alone, Diprovocim alone, or OVA alone (FIG. 3B and FIG. 4C). Relative to OVA alone, immunization with Diprovocim+OVA modestly but significantly slowed tumor growth rate but failed to prolong survival; a similar effect was observed with OVA immunization combined with anti-PD-L1 treatment (FIG. 3B and FIG. 4C).

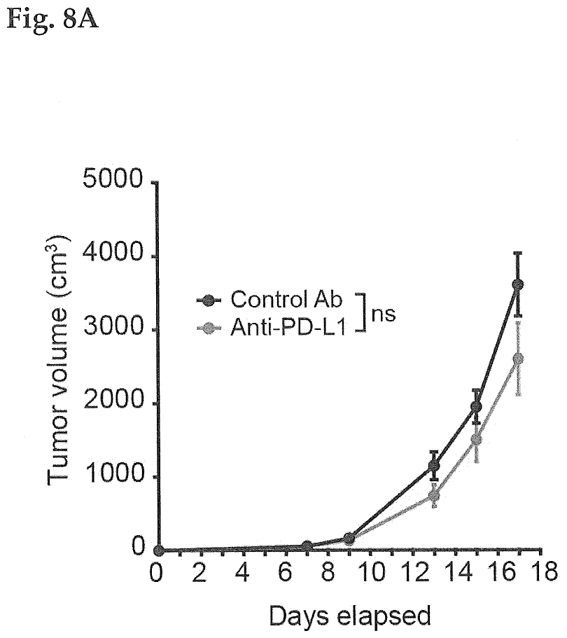

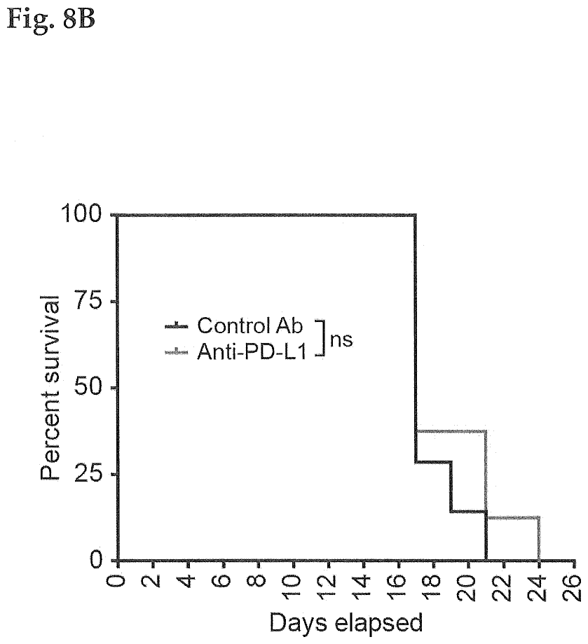

Strikingly, although without effect by itself (FIG. 8A and FIG. 8B) when anti-PD-L1 treatment was added to Diprovocim+OVA immunization, there was complete inhibition of tumor growth and 100% survival through eight weeks of observation (FIG. 3B and FIG. 4C). This dramatic antitumor effect was dependent on OVA immunization since Diprovocim alone combined with anti-PD-L1 treatment had no effect on tumor growth or mouse survival (FIG. 3D and FIG. 4E); this finding is consistent with poor immunogenicity of B16 melanoma (19-21).

To determine whether surviving mice were endowed with specific and long-term memory directed against the cancer antigen, the five-week survivors from FIG. 3C were re-challenged with B16-OVA cells and B16 cells lacking OVA (B16). In the absence of any further therapy, complete failure of B16-OVA tumor growth was observed, whereas B16 tumors grew rapidly (FIG. 3F). Both B16 and B16-OVA tumor cells grew at similar rates in naïve C57BL/6J mice (FIG. 3F).

Taken together, these data indicate that when used as an adjuvant in a cancer vaccine, a Diprovocim promotes antigen-specific antitumor immunity, which is greatly enhanced when combined with T cell checkpoint blockade in mice. Immunization with a Diprovocim as an adjuvant produces antigen-specific memory responses that protect the host from relapse of tumor growth.

The antitumor effect of a Diprovocim was examined in therapeutic immunization of mice with already established B16-OVA tumors. C57BL/6J mice were immunized with OVA with or without a Diprovocim on the day of or three days after tumor inoculation and received a booster immunization seven days later (FIG. 3A). In some mice, alum was substituted for Diprovocim to permit direct comparison between these two adjuvants. For all conditions, anti-PD-L1 treatment was initiated on day 3 after tumor inoculation and repeated every three days thereafter for 12 days.

Mice immunized with OVA alone on the day of tumor inoculation survived an average of 24 days, and 100% of mice (8/8) died by 38 days after tumor inoculation. As expected, Diprovocim+OVA administered on the day of tumor inoculation completely inhibited tumor growth, permitting 100% of mice (8/8) to survive through 54 days of observation (FIG. 3G and FIG. 4H). When alum was used instead of a Diprovocim in the same study, tumor growth was partially inhibited, and the average survival time was 37 days, with 25% of mice (2/8) surviving past 54 days (FIG. 3G and FIG. 4H).

When immunization was delayed until three days after tumor inoculation, Diprovocim+OVA still significantly inhibited tumor growth and prolonged average survival compared to OVA alone (41 days vs. 22 days) (FIG. 31 and FIG. 4J). In contrast, alum+OVA inhibited tumor growth but only slightly increased average survival time (30 days); the survival curve for alum+OVA was not significantly different from the curve for OVA alone (P=0.082) (FIG. 31 and FIG. 4J). In pre- and post-tumor treatment, the effect of Diprovocim on tumor growth, survival rate, and survival time was superior to that of alum.

Diprovocim Enhances Antitumor CTL Responses

The cellular mechanism by which combined anti-PD-L1 treatment and Diprovocim-adjuvanted immunization eliminates tumors was examined. Tumor-infiltrating leukocytes (TILs) of mice immunized with Diprovocim+OVA or alum+OVA were analyzed three days after tumor inoculation (FIG. 4A). Tumors were collected 14 days after inoculation, and single-cell suspensions were antibody stained and analyzed by flow cytometry to detect total leukocytes, CD4 and CD8 T cells, NK cells, DCs, and macrophages. The leukocytes were also stained with antibody to the H-2Kb MHC-class I tetramer bound to the OVA peptide (residues 257-264), as well as antibody against CD8 to identify tumor-specific CD8 T cells.

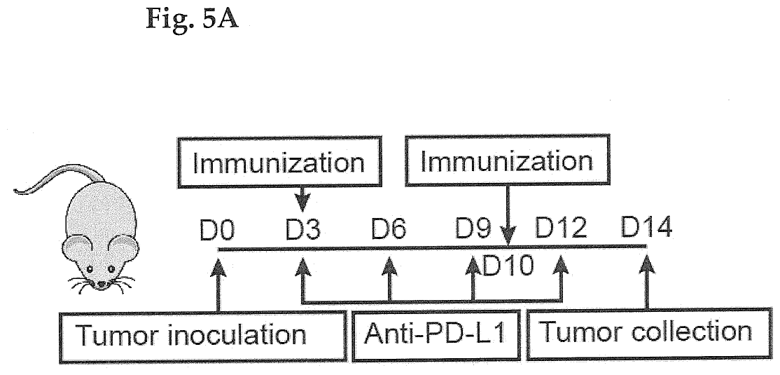

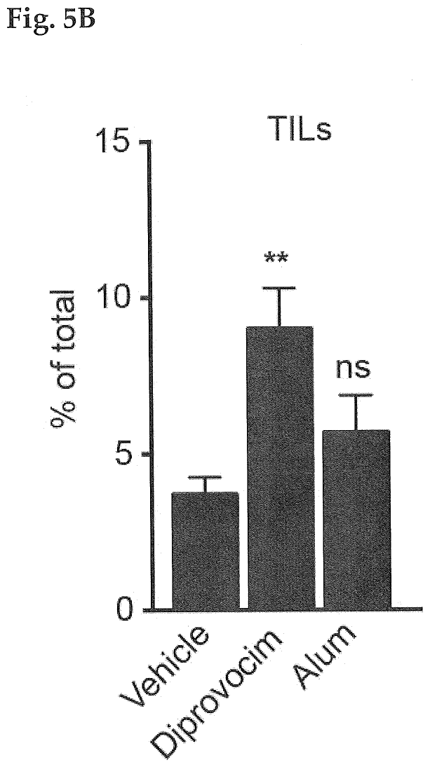

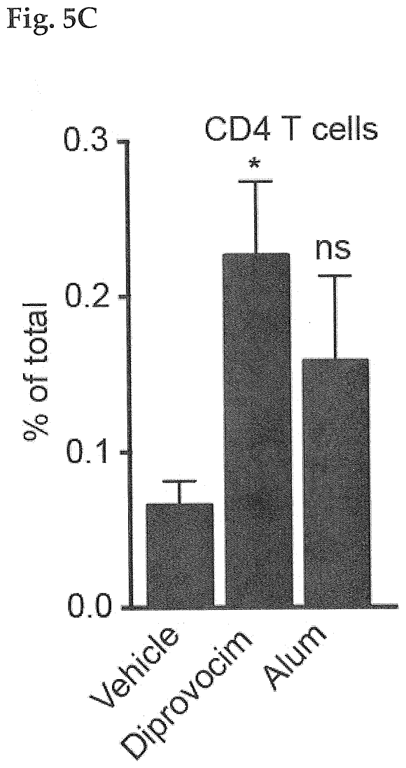

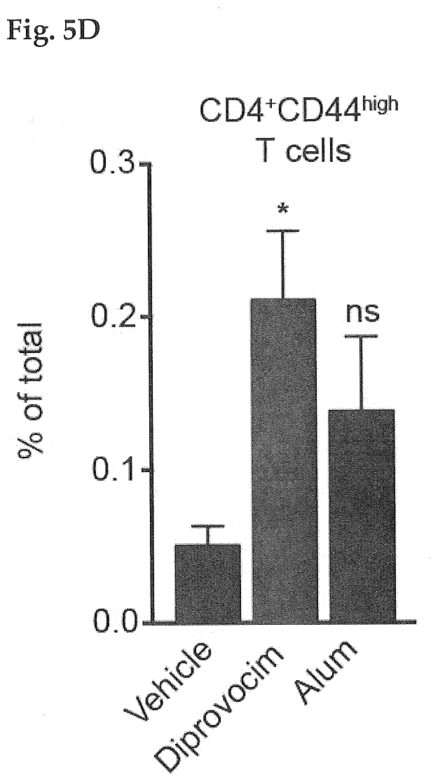

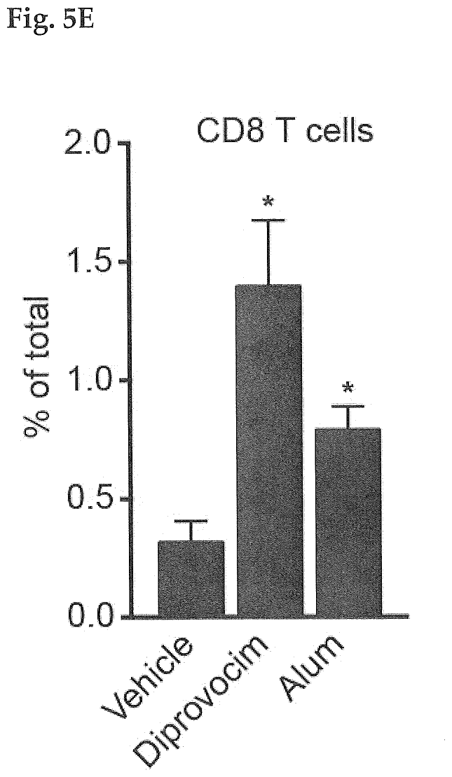

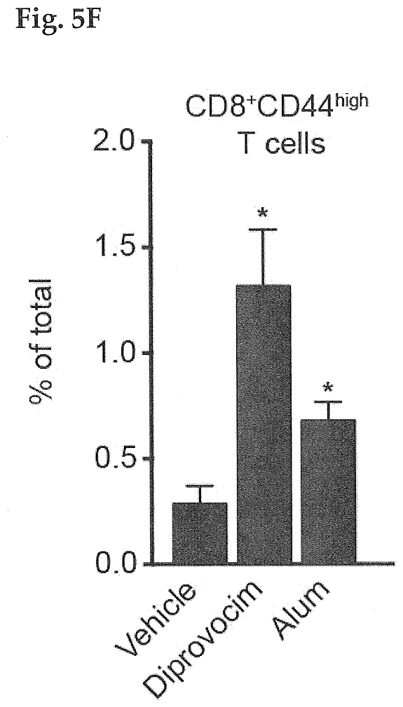

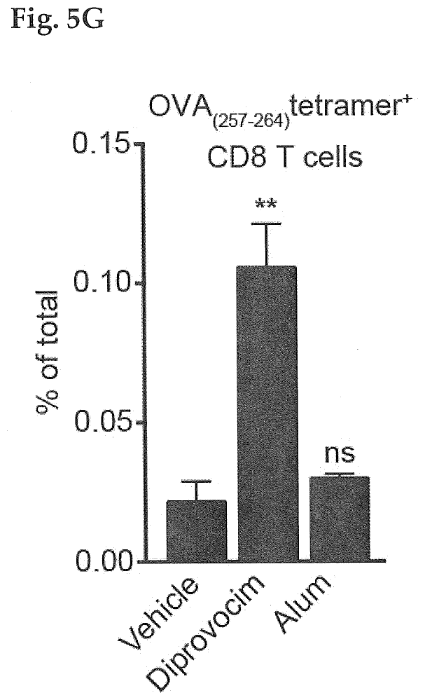

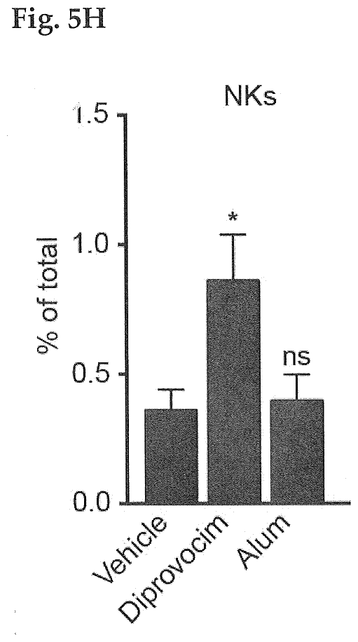

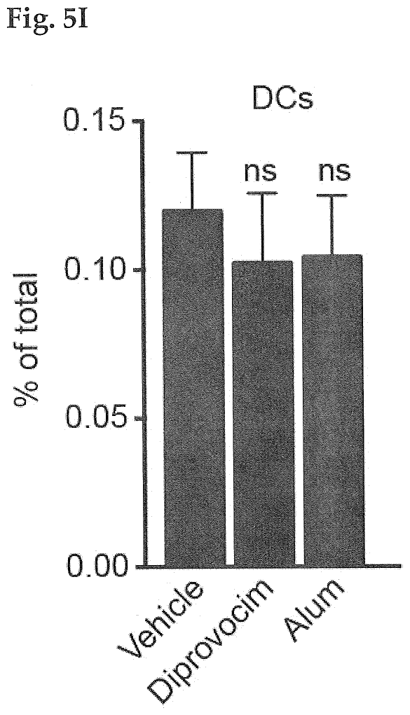

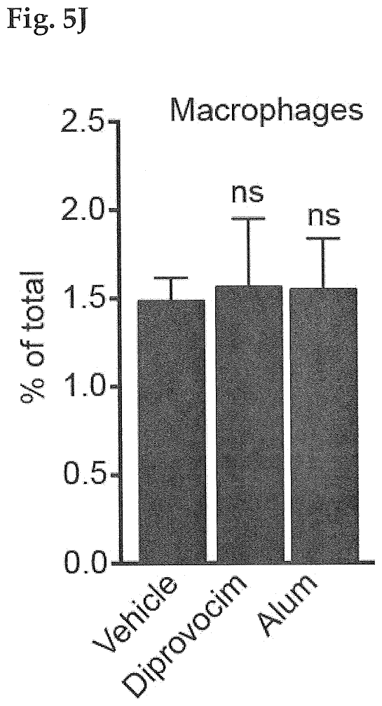

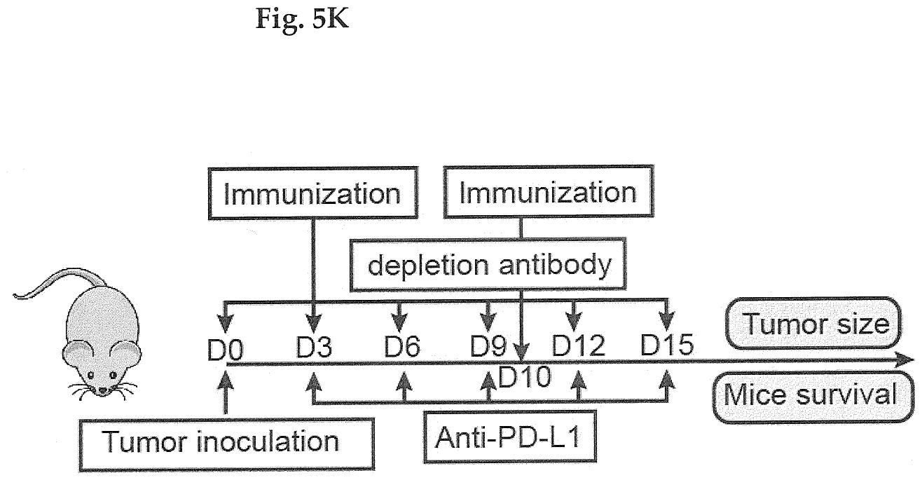

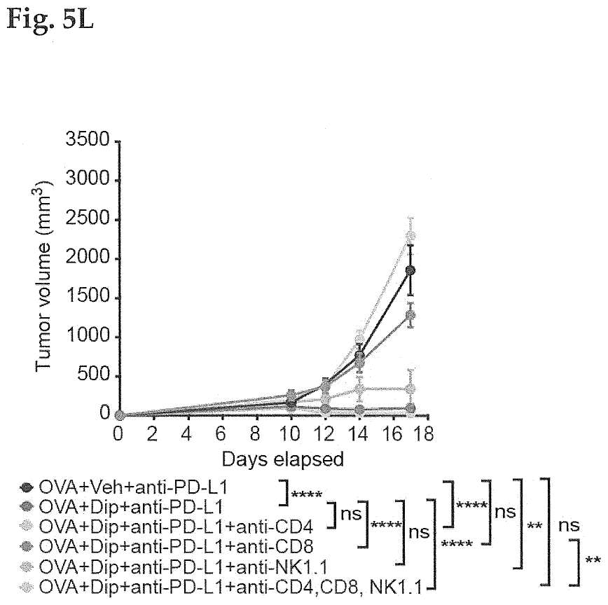

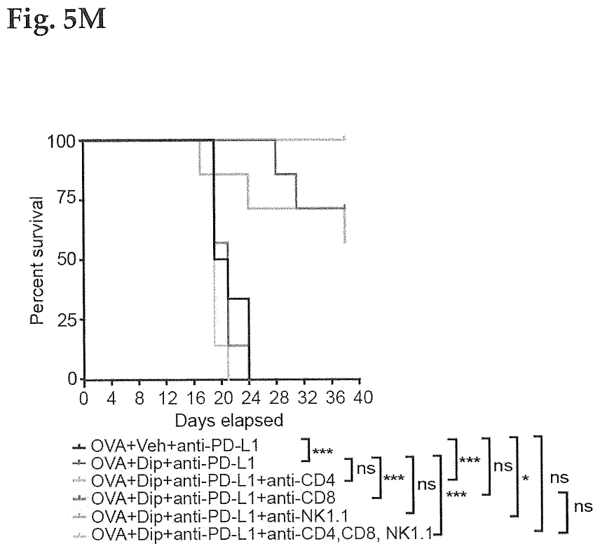

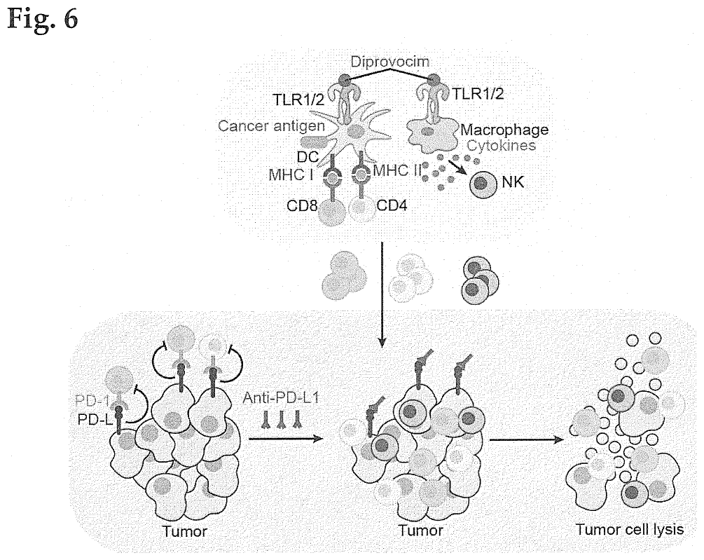

OVA immunizations containing a Diprovocim significantly increased the frequency of leukocytes in tumors compared to vehicle+OVA (FIG. 4B). Further analysis of these TILs revealed that a Diprovocim increased the frequencies of CD4 and CD8 T cells including activated CD4 and CD8 T cells (CD44^{high}) and OVA-specific CD8 T cells, as well as the frequency of NK cells (FIG. 4C—FIG. 5H).

Alum+OVA immunization showed a trend towards increasing TILs (FIG. 4B), which reached statistical significance for total and CD44^{high} CD8 T cells (FIG. 4E and FIG. 5F). However, the magnitude of the increase was reduced compared to that induced by Diprovocim+OVA. OVA-specific CD8 T cells were not increased by alum+OVA immunization (FIG. 4G) on day 14 after tumor inoculation; neither were total and CD44^{high} CD4 T cells (FIG. 4C and FIG. 5D), nor NK cells compared to vehicle+OVA (FIG. 4H).

The frequencies of intra-tumor DCs and macrophages were similar for mice immunized with vehicle+OVA, Diprovocim+OVA, and alum+OVA (FIG. 4I and FIG. 5J). Overall, these data indicate that the intra-tumor frequencies of CD4 and CD8 T cells, activated CD4 and CD8 T cells, OVA-specific CD8 T cells, and NK cells, but not DCs or macrophages, correlated with the antitumor effects of Diprovocim and alum in immunized mice.

To determine the immune cell population(s) necessary for the antitumor effect of Diprovocim+OVA plus anti-PD-L1, mice were depleted of CD8 T cells, CD4 T cells, NK cells, or all three cell populations using cell type-specific antibodies. The depletion antibodies were administered i.p. on the day of B16-OVA tumor inoculation (day 0) and every three days thereafter for 15 days (FIG. 4K). The effect of Diprovocim+OVA on both tumor growth and mouse survival was abrogated when mice were depleted of CD8 T cells or all three cell types together (CD4 T, CD8 T, NK cells) (FIG. 4L and FIG. 5M). In contrast, depletion of CD4 T cells or NK cells had little effect on the anti-tumor activity of Diprorocim+OVA (FIG. 4L and FIG. 5M).

Interestingly, a slight, statistically significant difference between the effects of CD8 T cell depletion vs. CD4+CD8+NK cell depletion was noted on tumor growth in mice treated with Diprovocim+OVA plus anti-PD-L1, in which tumor growth was greater in mice depleted of all three cell types. However, this difference did not translate to a difference in either survival rate or time. This finding suggests a minor role of either CD4 T cells, NK cells, or both, in mediating the anti-tumor effects of Diprovocim+OVA plus anti-PD-L1. These data demonstrate that CD8 T cells are necessary for tumor eradication by therapeutic Diprovocim+OVA immunization and checkpoint inhibition in mice.

### Discussion

It is believed that cancer vaccines targeted to tumor neoantigens can boost the success of immune checkpoint inhibition for cancer treatment by increasing the number and activation of tumor-specific CTLs capable of responding to checkpoint inhibitors. However, the type and magnitude of the T cell response to immunization depends critically on the vaccine adjuvant; currently only few adjuvants are approved for use in humans.

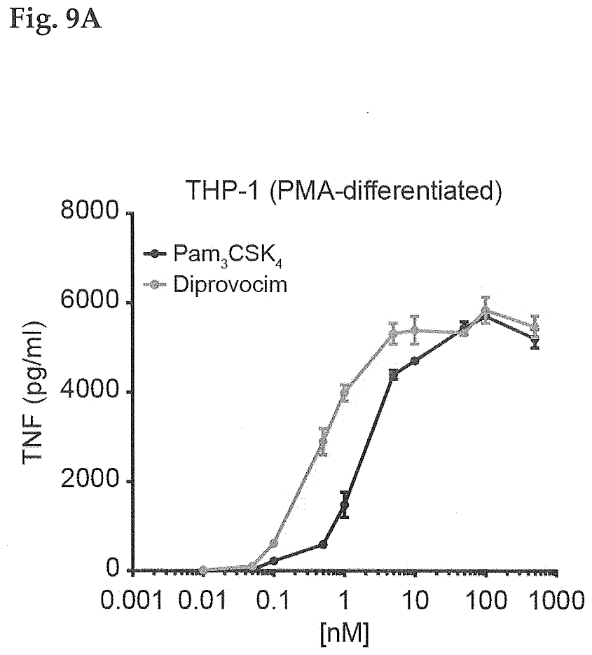

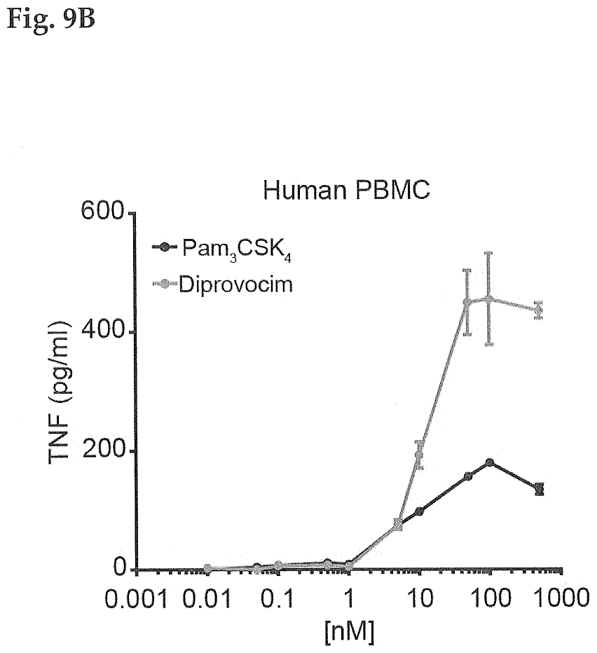

Here, the actions of a novel and potent adjuvant, Diprovocim, that engages and activates human and mouse TLR1/TLR2 heterodimers are described. A Diprovocim bears no structural similarity to other reported synthetic chemical ligands, nor to the natural ligands that activate TLR1/TLR2 (22-26). A Diprovocim is more potent and efficacious in activating human TLR1/TLR2 than Pam₃CSK₄ (FIG. 9A and FIG. 9B), a well-known ligand.

In mice, a Diprovocim induces strong TLR1- and TLR2-dependent humoral and CTL responses to a co-administered antigen. When combined with checkpoint inhibition, a Diprovocim-adjuvanted immunization causes antigen-specific eradication of a rapidly fatal tumor, and induces memory responses capable of preventing tumor regrowth. Cure of the tumor is observed despite the fact that checkpoint inhibition alone is insufficient to prevent a fatal outcome (FIG. 8A and FIG. 8B), supporting the premise of this combination immunotherapy.

The data support the following key mechanistic events mediating the antitumor effect of Diprovocim-adjuvanted immunization plus checkpoint inhibition (FIG. 5). Diprovocim binds to TLR1/TLR2 on APCs, activating them to produce pro-inflammatory cytokines and take up the administered tumor-specific antigens for processing and presentation via MHC I and MHC II. Antigen presentation, costimulatory molecule expression, and cytokine secretion by APCs induce proliferation and activation of antigen-specific CD4 T cells and CD8 T cells, which develop cytolytic activity toward tumor cells. NK cells are also activated by pro-inflammatory cytokines and infiltrate the tumor site. The addition of anti-PD-L1 inhibits the major immunosuppressive mechanism active in the tumor microenvironment, permitting uninhibited T cell activation and proliferation in response to TCR/CD28 ligation (27-29), further promoting tumor cell lysis mediated by CD8 T cells.

Numerous reports document both pro- and anti-tumorigenic effects of TLR2 signaling, which may depend on the cell type or type of cancer under study. For example, TLR2 signaling supports tumor growth through induction of immune suppressive cytokines such as IL-10, and activation of myeloid derived suppressor cells and tumor associated macrophages (30-32). In contrast, TLR2 signaling also promotes tumor regression by stimulating DC activation and cross-presentation (33), and downregulating Treg function (34-36).

For a Diprovocim, the overall outcome of systemic TLR2 activation, in the context of OVA immunization combined with immune checkpoint inhibition, was tumor cell lysis and tumor growth inhibition mediated by tumor-infiltrating antigen-specific CD8 T cells.

The therapeutic index of an adjuvant presumably depends upon the efficiency of conjoint targeting of antigen to an APC, and activation of that APC. The mode of interaction between a Diprovocim and TLR2 has been studied by X-ray crystallography, and its contacts with this subunit of the receptor will be reported elsewhere. The structure of a Diprovocim-TLR1/2 complex points to opportunities for a Diprovocim modification to incorporate immunogenic peptides, which might permit optimization of the therapeutic index by assuring that all active Diprovocim molecules are accompanied by antigen. Diprovocim is easy to synthesize and can be rapidly adapted to incorporate tumor-associated antigens and neoantigens. These features make it an attractive candidate for clinical development.

Materials and Methods

Mice

C57BL/6J, Tlr2^{-/-}, Myd88^{-/-} and OT-I mice were purchased from The Jackson Laboratory. Ly96^{-/-} (MD-2^{-/-}) mice were from RIKEN BRC. Tlr4^{1ps3/lps3}, Tlr6^{int/int}, Tlr7^{rsql/rsql}, Tirapt^{tor/tor}, Ticam1^{Lps2/Lps2}, Ticam1^{Lps2/Lps2}/Irak4^{otiose/otiose} mice were generated on a pure C57BL/6J background by ENU mutagenesis and are described at http://mutagenetix.utsouthwestern.edu.

Tlr1^{-/-} mice were created by CRISPR/Cas 9 gene targeting. Female C57BL/6J mice were superovulated by injection of 6.5 U pregnant mare serum gonadotropin (PMSG; Millipore), followed by injection of 6.5 U human chorionic gonadotropin (hCG; Sigma-Aldrich) 48 hours later. The superovulated mice were subsequently mated overnight with C57BL/6J male mice. The following day, fertilized eggs were collected from the oviducts and in vitro-transcribed Cas9 mRNA (50 ng/μl) and Tlr1 small base-pairing guide RNA (50 ng/μl; 5′-CAAACCGATCGTAGTGCTGA-3′; SEQ ID NO: XX) were injected into the cytoplasm or pronucleus of the embryos. The injected embryos were cultured in M16 medium (Sigma-Aldrich) at 37° C. in 5% CO₂. For the production of mutant mice, two-cell stage embryos were transferred into the ampulla of the oviduct (10-20 embryos per oviduct) of pseudo-pregnant Hsd:ICR (CD-1) female mice (Harlan Laboratories).

All experimental procedures using mice were approved by the Institutional Animal Care and Use Committee (IACUC) of the University of Texas Southwestern Medical Center, and were conducted in accordance with institutionally approved protocols and guidelines for animal care and use. All the mice were maintained at the University of Texas Southwestern Medical Center in accordance with institutionally approved protocols.

Isolation of Peritoneal Macrophages, BMDC, Human PBMC and Cell Culture

Thioglycollate-elicited macrophages were recovered 4 days after i.p. injection of 2 ml BBL thioglycollate medium, brewer modified (4% wt/vol; BD Biosciences) by peritoneal lavage with 5 ml phosphate buffered saline (PBS). The peritoneal macrophages were cultured in DMEM cell culture medium [DMEM containing 10% vol/vol FBS (Gemini Bio Products), 1% vol/vol penicillin and streptomycin (Life Technologies)] at 37° C. and 95% air/5% CO₂.

For murine BMDCs, bone morrow cells were cultured in Petri dishes in 10 ml DMEM cell culture medium containing 10 ng/ml of murine GM-CSF (R&D Systems). On day 3 of culture, this was replaced with fresh GM-CSF medium. Loosely adherent cells were transferred to a fresh Petri dish and cultured for an additional 4 d. Human PBMC were purchased from Stemcell Technologies. THP-1 (ATCC) cells were differentiated by treatment with 100 nM PMA (Sigma) in RPMI cell culture medium [RPMI containing 10% vol/vol FBS (Gemini Bio Products), 1% penicillin and streptomycin (Life Technologies)] for 24 hours. After that, cells were washed with PBS and cultured in fresh RPMI cell culture medium for 24 hours before use in studies.

Measurement of Cytokine Production

Cells were seeded onto 96-well plates at 1×10⁵ cells per well and stimulated with a Diprovocim (dissolved in DMSO, and final DMSO concentrations (0.2%) were kept constant in all experiments) for 4 h. Mouse TNF, IL-6, or IFN-β, or human TNF in the supernatants were measured by ELISA kits according to the manufacturer's instructions (eBioscience and PBL Assay Science). Pretreatment with 20 μg/ml anti-TLR1, anti-TLR2 or isotype control antibody (eBioscience) was for 1 hour. Unless otherwise indicated, mouse cells were from wild type C57BL/6J mice.

Western Blotting

Mouse peritoneal macrophages or human THP-1 cells (1×10⁶ per well) were stimulated in 12-well plates with a Diprovocim at 500 nM for mouse cells or 5 nM for human cells for the indicated times and lysed directly in sample buffer (Sigma). Cell lysates were separated by SDS-PAGE and transferred to nitrocellulose membranes. Membranes were probed with the following antibodies: phospho-IKKα (Ser176)/IKKβ (Ser177), IκBα, phospho-p38 (Thr180/Tyr182), phospho-JNK (Thr183/Tyr185), phospho-ERK1/2 (Thr202/Tyr204) (Cell Signaling Technology) and β-Actin (Sigma).

Immunization and Measurement of Antibody Response

EndoFit ovalbumin (OVA) with 98% purity minimum (SDS-PAGE) and endotoxin levels <1 EU/mg was purchased from Invivogen. Mice (4 mice per group) were immunized i.m. with 100 μg OVA mixed with vehicle (DMSO:Tween 80:saline=1:1:8), with 10 mg/kg Diprovocim, or with 2 mg/kg Alum (Alhydrogel adjuvant 2%, Invivogen). After 14 days, serum titers of OVA-specific IgG, IgG1, or IgG2b (SouthernBiotech) were measured by ELISA.

In Vivo CTL Killing Assay

C57BL/6J male mice were injected i.m. with 100 μg OVA plus 10 mg/kg Diprovocim (n=4 mice per group). One week later, naive C57BL/6J mice were killed, and splenocytes were collected. One-half of the splenocytes were left unpulsed, and half were pulsed with OVA257-263 peptides for 2 hours in complete medium [RPMI containing 10% vol/vol FBS, 1% penicillin and streptomycin] at 37° C. The unpulsed and peptide-pulsed cells were labeled, respectively, with 0.5 μM (“low”) or 5 μM (“high”) CellTrace Violet (Invitrogen) in serum-free medium for 20 minutes. Equal numbers (2×10⁶) of CellTrace Violet^{high} (OVA pulsed) and CellTrace Violet^{low} (unpulsed) cells were mixed together and injected intravenously into the immunized mice. After 48 hours, blood from treated mice was collected and subjected to flow cytometry analysis. The numbers of remaining live CellTrace Violet^{high} and CellTrace Violet^{low} cells were determined and used to calculate the percentage of OVA peptide-pulsed target cells killed. Specific killing was defined as

the ratio=CellTrace Violet^{low} cells/CellTrace Violet^{high} cells.

The percentage of

target cell lysis=[1−unimmunized ratio/immunized ratio]×100.

Tumor Inoculation, Immunization, and Tumor Measurement

B16-OVA cells (B16F10 melanoma cells stably expressing chicken ovalbumin) were grown in DMEM containing 10% vol/vol FBS. A total of 2×10⁵ B16-OVA cells in 100 μL PBS were injected s.c. into the right flank of 8-12 week old male C57BL/6J mice to establish tumors (n=8 mice per group). For pretreatment, 10 mg/kg Diprovocim-1 or 2 mg/kg alum with or without OVA (100 μg) was injected i.m. into mice on the same day as tumor inoculation (day 0). Mice received a booster shot seven days after the first immunization. On day 3, 6 and 9, some groups were injected i.p. with 200 μg checkpoint inhibitor (anti-mPD-L1, BioXcell) in 100 μl saline.

For post-treatment, 10 mg/kg Diprovocim-1 or 2 mg/kg Alum with OVA (100 μg) was injected i.m. into mice on day 3 after tumor inoculation. Mice received a booster shot seven days after the first immunization. On day 3, 6, 9, 12 and 15 after tumor inoculation, mice were also injected i.p. with 200 μg anti-mPD-L1 in 100 μl saline.

For depletion of CD4 T cells, CD8 T cells, and/or NK cells, 300 μg anti-mCD4 (BioXcell), 300 μg anti-mCD8 (BioXcell), 300 μg anti-mNK1.1 (BioXcell), or the three antibodies together in 200 μl saline were injected i.p. into mice on day 0, 3, 6, 9, 12, and 15 after tumor inoculation. 10 mg/kg Diprovocim with OVA (100 μg) or vehicle was injected i.m. into mice on day 3 after tumor inoculation. Mice received a booster shot seven days after the first immunization. On day 3, 6, 9, 12 and 15 after tumor inoculation, mice were also injected i.p. with 200 μg anti-mPD-L1 in 100 μl saline.

Tumors were measured with a digital caliper (Fisher) and the tumor sizes were calculated using the following formula: volume=0.5×length×width². Mice were sacrificed when the tumor length or width reached 2 cm.

Tumor-Infiltrating Leukocyte Separation and Staining

A total of 2×105 B16-OVA cells in 100 μL PBS were injected s.c. into the flank of each mouse to establish tumors (n=6 mice per treatment). 10 mg/kg Diprovocim-1 or 2 mg/kg alum with OVA (100 μg) was injected i.m. into mice on day 3 after tumor inoculation. Mice received a booster shot seven days after the first immunization. On day 3, 6, 9, and 12 after tumor inoculation, mice were also injected i.p. with 200 μg anti-mPD-L1.

On day 14 after tumor inoculation, tumors were harvested, minced and filtered through a 40-μm strainer to obtain single-cell suspensions. Red blood cells were lysed with RBC lysis buffer (Sigma). After pelleting, cells were stained with a mixture of antibodies for 45 minutes, including anti-mouse CD45.2-PE or CD45.2-APC (BioLegend), anti-mouse CD3-FITC (BD Biosciences), anti-mouse CD4-BV786 (BD Biosciences), anti-mouse CD8-BV510 (BioLegend), anti-mouse CD44-PE-CF594 (BioLegend), APC-conjugated H-2Kb/OVA (SIINFEKL; SEQ ID NO: XX) tetramer (Baylor College of Medicine), anti-mouse F4/80-PE (Tonbo Bioscience), anti-mouse CD11b-BV605 (BioLegend), anti-CD11c-BV711 (BD Biosciences), anti-NK1.1-BV650 (BD Biosciences). Then, the cells were washed twice with PBS. Stained cells were analyzed with an LSRII instrument (BD Biosciences) and the flow cytometry data were analyzed using FlowJo software.

Measurement of Cross-Priming of CD8 T Cells

C57BL/6J male mice were injected i.m. with 100 μg OVA mixed with vehicle or with 10 mg/kg Diprovocim (n=4 mice per treatment). 24 hours later, DCs from draining lymph nodes and spleen were purified by Mouse Pan Dendritic Cell Isolation Kit (Miltenyi Biotech). CD8 T cells from OT-I transgenic mice were purified by Mouse CD8+ T Cell Isolation Kit (Miltenyi Biotech). 3×10⁵ DCs were co-cultured with 3×10⁵ OT-I CD8 T cells in RPMI medium containing 10% vol/vol FBS and 1% vol/vol penicillin and streptomycin for 24 hours. Then, cells were collected and stained with anti-mouse CD3-FITC, anti-mouse CD8-BV510, and anti-mouse CD69-PE-CF594 (BioLegend) for 45 minutes. Then, the cells were washed twice with PBS. Stained cells were analyzed with an LSRII instrument and the flow cytometry data were analyzed using FlowJo software.

Statistical Analyses

Data represent means±SEM in all graphs depicting error bars. The statistical significance of differences between experimental groups was determined using GraphPad Prism 7 and the indicated statistical tests. For comparisons of differences between two unpaired experimental groups, an unpaired Student's t test was used and two-tailed P values are reported. P values are indicated by *P≤0.05; **P≤0.01; ***P≤0.001; ****P≤0.0001. P≤0.05 was considered statistically significant.

Methods

Synthesis of Diprovocim-1

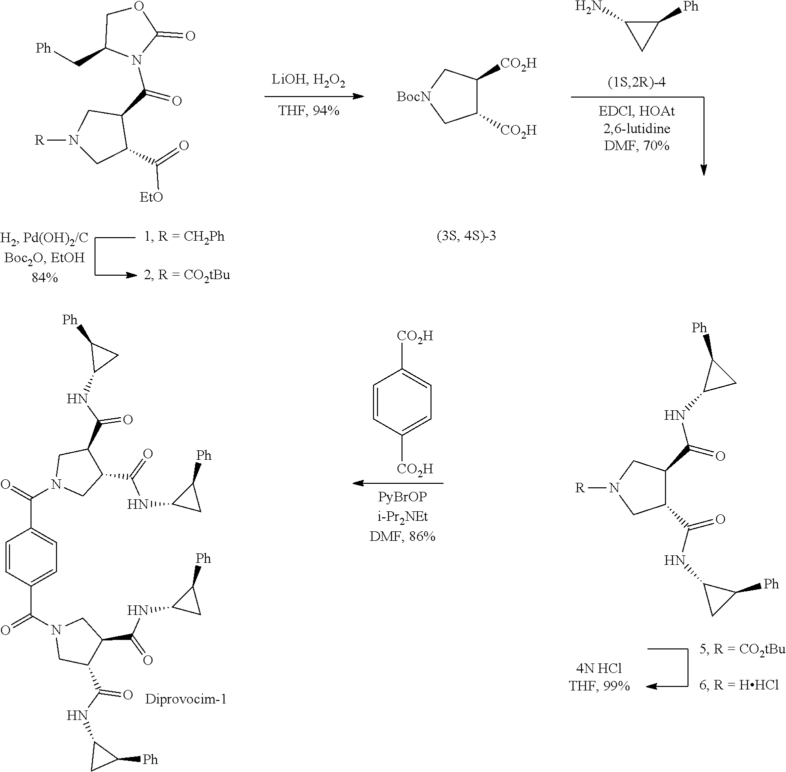

The syntheses of Diprovocim molecules and intermediates are shown and discussed in detail in WO 2018/005812 1A, published on Jan. 4, 2018. Illustrative syntheses are also set out hereinbelow.

(3S,4S)-1-tert-Butyl 3-Ethyl 4-((S)-4-Benzyl-2-oxooxazolidine-3-carbonyl)pyrrolidine-1,3-dicarboxylate (2). (3S,4S)-Ethyl 1-benzyl-4-((S)-4-benzyl-2-oxooxazolidine-3-carbonyl)pyrrolidine-3-carboxylate¹ (1, 3.43 g, 7.86 mmol) and Boc₂O (1.80 g, 8.25 mmol, 1.05 equiv) were dissolved in ethanol (EtOH, 50 mL) at room temperature. Pd(OH)₂/C (500 mg) was added and the reaction mixture was sparged with nitrogen (N₂) for 15 minutes. ¹ Prepared as a single stereoisomer according to: Bao et al., U.S. Pat. No. 6,489,354 B1.

A 3-way flushing adapter, equipped with a hydrogen (H₂) filled balloon and vacuum source, was attached. The headspace above the reaction mixture was evacuated until the solvent began to boil, then backfilled with H₂. This vacuum/fill process was repeated 10-15 times to maximize H₂ in the headspace. After stirring for 18 hours, the reaction mixture was filtered through a 6 cm Celite plug, rinsing with EtOH aliquots (3×15 mL) thoroughly, and concentrated. Flash column chromatography (SiO₂, 25% EtOAc/hexanes) provided 2.93 g (84%) of 2 as a clear, viscous oil.

¹H NMR (400 MHz, CDCl₃) δ 7.40-7.19 (m, 5H), 4.69 (dd, J=9.0, 4.5 Hz, 1H), 4.52 (q, J=7.7 Hz, 1H), 4.29-4.14 (m, 4H), 3.95-3.75 (m, 2H), 3.60 (m, 2H), 3.52-3.27 (m, 2H), 2.86-2.71 (m, 1H), 1.46 (s, 9H), 1.28 (t, J=7.5 Hz, 3H). HRMS (ESI-TOF) m/z calcd for C₂₃H₃₁N₂O₇ [M+H]+447.2126, found 447.2126.

(3S,4S)-1-(tert-Butoxycarbonyl)pyrrolidine-3,4-dicarboxylic Acid (3).² (3S,4S)-1-tert-Butyl 3-ethyl 4-((S)-4-benzyl-2-oxooxazolidine-3-carbonyl)-pyrrolidine-1,3-dicarboxylate ((3S,4S)-2, 2.06 g, 4.63 mmol) was dissolved in anhydrous tetrahydrofuran (THF, 20 mL) and cooled to 0° C. Hydrogen peroxide (2.10 mL, ca. 18.5 mmol, 4.0 equiv, 30% w/v) was added dropwise to the stirred reaction solution. After 3-5 minutes, LiOH.H₂O (500 mg, 11.9 mmol) was added. After 2 hours, additional LiOH (470 mg, 11.2 mmol) was added, along with H₂O (10 mL) and THF (15 mL). ² Modified procedure from: Ma et al., Tetrahedron Asymm. 8, 883-887 (1997).

The aqueous THF reaction mixture was stirred 3 hours, warming to room temperature. Saturated aqueous Na₂SO₃ (10 mL) was added, and the THF was removed under a N₂ stream. The resulting mixture was poured into H₂O (200 mL) and extracted with methylene chloride (CH₂Cl₂, 2×100 mL) to remove the oxazolidinone.

The aqueous phase was acidified with the addition of aqueous 1N HCl to pH 2 (ca. 75 mL). The aqueous phase was extracted with ethyl acetate (EtOAc, 3×125 mL), and the organic extracts were dried over Na₂SO₄, filtered and concentrated to provide 1.13 g (94%) of (S,S)-3 as a white solid. ¹H NMR (500 MHz, DMSO-d₆) δ 3.59-3.48 (m, 2H), 3.41-3.31 (m, 2H), 3.30-3.18 (m, 2H), 1.39 (s, 9H).

(3S,4S)-tert-Butyl 3,4-Bis(((1S,2R)-2-phenylcyclopropyl)carbamoyl)pyrrolidine-1-carboxylate (5). (3S,4S)-1-(tert-Butoxycarbonyl)pyrrolidine-3,4-dicarboxylic acid ((S,S)-3, 775 mg, 2.99 mmol), (1S,2R)-trans-2-phenylcyclopropylamine ((1S,2R)-4, 816 mg, 6.13 mmol, 2.05 equiv, commercially available from D-L Chiral Chemicals), and 1-hydroxy-7-azabenzotriazole (HOAt, 895 mg, 6.58 mmol, 2.20 equiv) were dissolved in anhydrous dimethylformamide (DMF, (15 mL) under a N₂ atmosphere. 2,6-Lutidine (1.75 mL, 14.9 mmol, 5.00 equiv) was added slowly. Upon dissolution of the reagents (about 15 minutes), 1-ethyl-3-(3-dimethylaminopropyl)carbodiimide hydrochloride (EDCI.HCl, 1.43 g, 7.47 mmol, 2.50 equiv) was added in one portion, and the reaction mixture was stirred for 18 hours, after which it was poured into aqueous 1N HCl (150 mL) and EtOAc (100 mL).

The aqueous phase was extracted with EtOAc (2×75 mL), and the combined organic phases were washed with aqueous 1N HCl (75 mL), saturated aqueous NaHCO₃ (75 mL), and saturated aqueous NaCl (50 mL) sequentially. The organic phase was dried over Na₂SO₄, filtered and concentrated. Flash column chromatography (SiO₂, 50% EtOAc/hexanes) provided 1.02 g (70%) of 5.

¹H NMR (400 MHz, CDCl₃) δ 7.33-7.24 (m, 5H), 7.23-7.09 (m, 5H), 6.61 (s, 1H), 6.43 (s, 1H), 3.85 (t, J=9.7 Hz, 1H), 3.68 (m, 1H), 3.60 (t, J=10.5 Hz, 1H), 3.42 (t, J=10.4 Hz, 1H), 3.27 (q, J=10.0, 9.3 Hz, 1H), 3.12 (t, J=9.7 Hz, 1H), 2.88 (m, 2H), 2.05 (ddt, J=9.8, 6.4, 3.4 Hz, 2H), 1.46 (s, 9H), 1.24 (q, J=6.6 Hz, 2H), 1.13 (dt, J=10.1, 5.3 Hz, 2H). HRMS (ESI-TOF) m/z calcd for C₂₉H₃₆N₃O₄ [M+H]+490.2700, found 490.2705.

(3S,4S)—N³,N⁴-Bis((1S,2R)-2-phenyl-cyclopropyl)pyrrolidine-3,4-dicarboxamide Hydrochloride (6). (3S,4S)-tert-Butyl 3,4-bis(((1S,2R)-2-phenylcyclopropyl)carbamoyl)-pyrrolidine-1-carboxylate (5, 998 mg, 2.04 mmol) was suspended in anhydrous THF (2 mL) at room temperature. 4N HCl (8 mL, 4.0 M solution in dioxane) was added dropwise to the vigorously stirred reaction solution. After stirring 3 hours at room temperature, during which some product had precipitated from the reaction mixture, the solvents were removed by N₂ stream over 16 hours. The residual solids were suspended in anhydrous THF and reconcentrated in vacuo (3×5 mL) to ensure complete removal of the dioxane and excess HCl. This process was repeated with anhydrous Et₂O (3×5 mL) to provide 870 mg (99%) of 6 as an amorphous white solid.

¹H NMR (500 MHz, DMSO-d₆) δ 9.35 (s, 2H), 8.76 (d, J=4.4 Hz, 2H), 7.26 (t, J=7.6 Hz, 4H), 7.20-7.06 (m, 6H), 3.76-3.62 (m, 1H), 3.55-3.42 (m, 1H), 3.26 (t, J=8.2 Hz, 2H), 3.21-3.11 (m, 2H), 2.90-2.78 (m, 2H), 1.99 (ddd, J=9.6, 6.3, 3.4 Hz, 2H), 1.26-1.13 (m, 4H). HRMS (ESI-TOF) m/z calcd for C₂₄H₂₈N₃O₂ [M+H]⁺390.2176, found 390.2178.

Diprovocim-1: (3S,3'S,4S,4'S)-1,1′-Terephthaloylbis(N³,N⁴-bis((1S,2R)-2-phenylcyclo-propyl)pyrrolidine-3,4-dicarboxamide). (3S,4S)—N³,N⁴-Bis((1S,2R)-2-phenylcyclopropyl)-pyrrolidine-3,4-dicarboxamide hydrochloride (6, 500 mg, 1.17 mmol, 2.20 equiv) and terephthalic acid (benzene-1,4-dicarboxylic acid, 89 mg, 0.53 mmol, 1.00 equiv) were dissolved in anhydrous DMF (6 mL) at room temperature. i-Pr₂NEt (0.280 mL, 1.60 mmol, 3.00 equiv) was added, followed by bromo-tris-pyrrolidinophosphonium hexafluorophosphate (PyBrOP, 497 mg, 1.07 mmol, 2.00 equiv) after 5 minutes and the mixture was stirred at 23° C. for 18 hours. After 18 hours, the reaction mixture was diluted with EtOAc (300 mL) and washed with aqueous 0.5N HCl (2×150 mL). The aqueous phase was extracted with EtOAc (1×50 mL).

The combined organic phases were washed with saturated aqueous NaHCO₃ (100 mL) and saturated aqueous NaCl (75 mL). The organic phase was dried over Na₂SO₄, decanted and concentrated. Flash column chromatography (SiO₂, 5-8% MeOH/CH₂Cl₂) provided Diprovocim-1. Diprovocim-1 could be further purified by trituration with cold (0° C.) 1:1 Et₂O/EtOAc (3×5 mL), decanting off the liquid phase to provide 421 mg (86%) of pure diprovocim. [α]_{D}²⁶+57 (c 0.33, EtOH). IR (neat) vₘₐₓ 3259, 1633, 1539, 1426, 1386, 1073, 695 cm⁻¹.

¹H NMR (600 MHz, DMSO-d₆) δ 8.42 (d, J=4.3 Hz, 2H), 8.29 (d, J=4.3 Hz, 2H), 7.56 (s, 4H), 7.27-7.21 (m, 8H), 7.19-7.09 (m, 8H), 7.09-7.03 (m, 4H), 3.80 (dd, J=12.0, 8.6 Hz, 2H), 3.71-3.58 (m, 2H), 3.51 (ddd, J=15.6, 11.2, 8.2 Hz, 4H), 3.19 (q, J=8.4 Hz, 2H), 3.10 (q, J=8.1 Hz, 2H), 2.90-2.80 (m, 2H), 2.80-2.73 (m, 2H), 1.97 (ddd, J=9.6, 6.4, 3.4 Hz, 2H), 1.86 (ddd, J=9.5, 6.3, 3.4 Hz, 2H), 1.21-1.13 (m, 4H), 1.13-1.05 (m, 4H). ¹³C NMR (151 MHz, DMSO-d₆) δ 171.65, 170.93, 167.46, 141.28, 141.19, 137.71, 128.17, 128.14, 127.09, 125.83, 125.79, 125.60, 51.48, 48.74, 46.95, 45.83, 45.07, 32.54, 32.45, 25.87, 23.90, 23.81, 15.33, 15.24. HRMS (ESI-TOF) m/z calcd for C₅₆H₅₇N₆O₆ [M+H]⁺ 909.4334, found 909.4334.

### CITATIONS

1. Hou B, Reizis B & DeFranco A L (2008) Toll-like receptors activate innate and adaptive immunity by using dendritic cell-intrinsic and -extrinsic mechanisms. Immunity 29(2): 272-282.
2. MacLeod H & Wetzler L M (2007) T cell activation by TLRs: A role for TLRs in the adaptive immune response. Sci STKE 2007(402): pe48.
3. Coffman R L, Sher A & Seder R A (2010) Vaccine adjuvants: Putting innate immunity to work. Immunity 33(4): 492-503.
4. Haanen JBAG (2017) Converting cold into hot tumors by combining immunotherapies. Cell 170(6): 1055-1056.
5. Sharma P, Hu-Lieskovan S, Wargo J A & Ribas A (2017) Primary, adaptive, and acquired resistance to cancer immunotherapy. Cell 168(4): 707-723.
6. Wang H, et al (2017) cGAS is essential for the antitumor effect of immune checkpoint blockade. Proc Natl Acad Sci USA 114(7): 1637-1642.
7. Zou W, Wolchok J D & Chen L (2016) PD-L1 (B7-H1) and PD-1 pathway blockade for cancer therapy: Mechanisms, response biomarkers, and combinations. Sci Transl Med 8(328): 328rv4.
8. Topalian S L, Drake C G & Pardoll D M (2015) Immune checkpoint blockade: A common denominator approach to cancer therapy. Cancer Cell 27(4): 450-461.
9. Takeda Y, et al (2017) A TLR3-specific adjuvant relieves innate resistance to PD-L1 blockade without cytokine toxicity in tumor vaccine immunotherapy. Cell Rep 19(9): 1874-1887.
10. Shah R R, Hassett K J & Brito L A (2017) Overview of vaccine adjuvants: Introduction, history, and current status. Methods Mol Biol 1494: 1-13.
11. Reed S G, Orr M T & Fox C B (2013) Key roles of adjuvants in modern vaccines. Nat Med 19(12): 1597-1608.
12. Kawai T & Akira S (2010) The role of pattern-recognition receptors in innate immunity: Update on toll-like receptors. Nat Immunol 11(5): 373-384.
13. Kawasaki T & Kawai T (2014) Toll-like receptor signaling pathways. Front Immunol 5: 461.
14. Beutler B A (2009) TLRs and innate immunity. Blood 113(7): 1399-1407.
15. Lim K H & Staudt L M (2013) Toll-like receptor signaling. Cold Spring Harb Perspect Biol 5(1): a011247.
16. Wang S, et al (2016) Intratumoral injection of a CpG oligonucleotide reverts resistance to PD-1 blockade by expanding multifunctional CD8+ T cells. Proc Natl Acad Sci USA 113(46): E7240-E7249.
17. Dowling J K & Mansell A (2016) Toll-like receptors: The swiss army knife of immunity and vaccine development. Clin Transl Immunology 5(5): e85.
18. Goldberg J, et al (2002) Erythropoietin mimetics derived from solution phase combinatorial libraries. J Am Chem Soc 124(4): 544-555.
19. Ueha S, et al (2015) Robust antitumor effects of combined anti-CD4-depleting antibody and anti-PD-1/PD-L1 immune checkpoint antibody treatment in mice. Cancer Immunol Res 3(6): 631-640.
20. Lechner M G, et al (2013) Immunogenicity of murine solid tumor models as a defining feature of in vivo behavior and response to immunotherapy. J Immunother 36(9): 477-489.
21. Chen L, et al (1994) Tumor immunogenicity determines the effect of B7 costimulation on T cell-mediated tumor immunity. J Exp Med 179(2): 523-532.
22. Murgueitio M S, et al (2017) Enhanced immunostimulatory activity of in silico discovered agonists of toll-like receptor 2 (TLR2). Biochim Biophys Acta 1861(11 Pt A): 2680-2689.
23. Guo X, et al (2017) The novel toll-like receptor 2 agonist SUP3 enhances antigen presentation and T cell activation by dendritic cells. Front Immunol 8: 158.
24. Guan Y, Omueti-Ayoade K, Mutha S K, Hergenrother P J & Tapping R I (2010) Identification of novel synthetic toll-like receptor 2 agonists by high throughput screening. J Biol Chem 285(31): 23755-23762.
25. Cheng K, et al (2015) Specific activation of the TLR1-TLR2 heterodimer by small-molecule agonists. Sci Adv 1(3): 10.1126/sciadv.1400139.
26. Jin M S, et al (2007) Crystal structure of the TLR1-TLR2 heterodimer induced by binding of a tri-acylated lipopeptide. Cell 130(6): 1071-1082.
27. Freeman G J, et al (2000) Engagement of the PD-1 immunoinhibitory receptor by a novel B7 family member leads to negative regulation of lymphocyte activation. J Exp Med 192(7): 1027-1034.
28. Nishimura H, Nose M, Hiai H, Minato N & Honjo T (1999) Development of lupus-like autoimmune diseases by disruption of the PD-1 gene encoding an ITIM motif-carrying immunoreceptor. Immunity 11(2): 141-151.
29. Dong H, Zhu G, Tamada K & Chen L (1999) B7-H1, a third member of the B7 family, co-stimulates T-cell proliferation and interleukin-10 secretion. Nat Med 5(12): 1365-1369.
30. Tang M, et al (2015) Toll-like receptor 2 activation promotes tumor dendritic cell dysfunction by regulating IL-6 and IL-10 receptor signaling. Cell Rep 13(12): 2851-2864.
31. Yamazaki S, et al (2011) TLR2-dependent induction of IL-10 and Foxp3+CD25+CD4+ regulatory T cells prevents effective anti-tumor immunity induced by Pam2 lipopeptides in vivo. PLoS One 6(4): e18833.
32. Shime H, et al (2017) Toll-like receptor 2 ligand and interferon-gamma suppress anti-tumor T cell responses by enhancing the immunosuppressive activity of monocytic myeloid-derived suppressor cells. Oncoimmunology 7(1): e1373231.
33. Shen K Y, et al (2014) Molecular mechanisms of TLR2-mediated antigen cross-presentation in dendritic cells. J Immunol 192(9): 4233-4241.
34. Nyirenda M H, et al (2011) TLR2 stimulation drives human naive and effector regulatory T cells into a Th17-like phenotype with reduced suppressive function. J Immunol 187(5): 2278-2290.
35. Zhang Y, et al (2011) TLR1/TLR2 agonist induces tumor regression by reciprocal modulation of effector and regulatory T cells. J Immunol 186(4): 1963-1969.
36. Amiset L, et al (2012) TLR2 ligation protects effector T cells from regulatory T-cell mediated suppression and repolarizes T helper responses following MVA-based cancer immunotherapy. Oncoimmunology 1(8): 1271-1280.

Each of the patents, patent applications and articles cited herein is incorporated by reference.

The foregoing description and the examples are intended as illustrative and are not to be taken as limiting. Still other variations within the spirit and scope of this invention are possible and will readily present themselves to those skilled in the art.

1. A method of inhibiting the growth of diseased cells in a mammal, which diseased cells exhibit a marker molecule that is absent on cells of the same type that are free of said disease or is present on the disease-free cells in significantly reduced numbers compared to said diseased cells, that comprises the steps of:
a) administering to said mammal (i) an adjuvant-sufficient amount of a Diprovocim compound, (ii) a T cell-stimulating amount of an immune checkpoint inhibitor, and (iii) an immunizing amount of said marker molecule or portion thereof;
b) maintaining said immunized mammal for a time period sufficient for said mammal to mount an immune response to said immunization; and
wherein said Diprovocim compound corresponds in structure to structural Formula V, 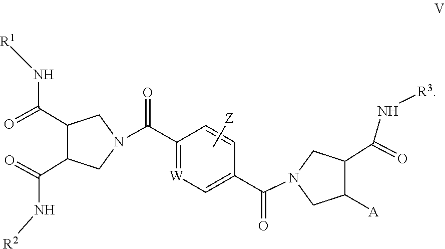
wherein
-A is —H (hydrido) or —C(O)NH—R⁴;
R¹, R², R³ and R⁴ are the same or different and are a 2-(4-fluorophenyl)ethyl, a trans-2-phenylcyclopropyl, a trans-2-(4-fluorophenyl)-cyclopropyl or a C₂-C₁₈ hydrocarbyl group with the provisos that:
1) at least two of R¹, R², R³ and R⁴ (R¹⁻⁴) or at least two of R¹, R², and R³ (R¹⁻³) are a trans-2-phenylcyclopropyl, a trans-2-(4-fluorophenyl)-cyclopropyl group or a mixture thereof, or each of R¹⁻⁴ is a 2-(4-fluorophenyl)ethyl group,
2) at least one depicted pyrrolidinyldicarboxamido group has the (S,S) configuration, and each depicted R substituent other than a C₂-C₁₈ hydrocarbyl group is a trans-2-phenylcyclopropyl, a trans-2-(4-fluorophenyl)-cyclopropyl group or a mixture thereof when each of R¹⁻⁴ is other than 2-(4-fluorophenyl)ethyl,
3) no more than two of R¹⁻⁴ are C₂-C₁₈ hydrocarbyl groups when -A is —C(O)NH—R⁴, and
4) when A is hydrido, one of R¹⁻³ can be a C2-C18 hydrocarbyl group and the depicted R³-containing pyrrolidinylcarboxamido group can have either the R or S configurations, or a mixture of both configurations;
—Z is one or more of halogen —H, —NH₂, —OH, —OCH₃, —NO₂, —OCH₂CO₂H, —O(CH₂CH₂O)ₙCH₂CH₂CO₂H, —OCH₂CONH(CH₂CH₂O)ₙCH₂CH₂CO₂H, —NHCOCH₂O—(CH₂CH₂O)ₙCH₂CO₂H, —OCH₂CONHCH₂CONHCH(CHOH)CO₂H, —OCH₂CONHCH₂CONHCHCO₂H(CH₂CO₂H), —OCH₂CONHCH₂CONHCH(CHOH) (CH₂CH₂O)ₙCH₂CH₂CO₂H, —OCH₂CONHCH₂CONHCH(CHOH) (CH₂CH₂O)ₙCH₂CH₂CO₂H, —OCH₂CONHCH₂CONHCH[(CH₂)₄NH₂]CO₂H, —OCH₂CONHCH₂CONHCH(CH₂OH)CO{NHCH[(CH₂)₄NH₂]CO}ₘNHCH—[(CH₂)₄NH₂]CO₂H (SEQ ID NOs: 3-8), —OCH₂CONHCH₂CO{NHCH[(CH₂)₄NH₂]CO}ₚNHCH[(CH₂)₄NH]CO₂H (SEQ ID NOs: 9-13) and —OCH₂CONHCH₂CO{NHCH(CH₂OH)CO}_{q}NHCH(CH₂OH)CO₂H (SEQ ID NOs: 14-18);
W is nitrogen (N) or CH;
“n” is a number whose average value is one to about eight;
“m” is a number whose value is 1 to about 6; “p” is a number whose value is 1 to about 6; and
“q” is a number whose value is 1 to about 6.

2. The method according to claim 1, wherein said Diprovocim compound, immune checkpoint inhibitor, and marker molecule are administered dissolved or dispersed in an immunizing pharmaceutical composition.

3. The method according to claim 2, wherein said Diprovocim compound, immune checkpoint inhibitor, and marker molecule or portion thereof are administered dissolved or dispersed in separate immunizing pharmaceutical compositions.

4. The method according to claim 2, wherein said Diprovocim compound and marker molecule or portion thereof are administered dissolved or dispersed from the same immunizing pharmaceutical composition.

5. The method according to claim 2, wherein said Diprovocim compound, immune checkpoint inhibitor, and marker molecule marker molecule or portion thereof are administered together dissolved or dispersed from the same immunizing pharmaceutical composition.

6. The method according to claim 2, wherein said immunizing composition is an aqueous medium.

7. The method according to claim 1, wherein said immune checkpoint inhibitor is a paratope-containing molecule.

8. The method according to claim 1, wherein the substituents of each depicted pyrrolidinyldicarboxamido group in Formula V has the (S, S) configuration.

9. The method according to claim 1, wherein W is CH, said Diprovocim compound has the structural Formula Ia, and wherein the R¹⁻⁴ and Z moieties are 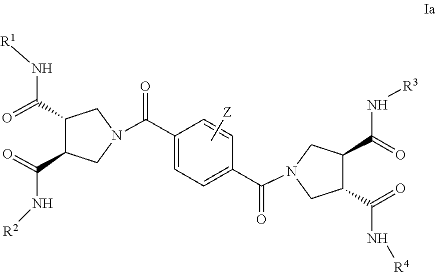
as described above.

10. The method according to claim 1, wherein —Z is —H.

11. The method according to claim 10, wherein each of R¹⁻⁴ is a trans-2-phenylcyclopropyl group or a trans-2-(4-fluorophenyl)cyclopropyl group having the (1S,2R) configuration.

12. The method according to claim 1, wherein said diseased cells are cancerous or pathogen-infected.

13. The method according to claim 12, wherein said diseased cells are cancerous solid tumor cells.

14. The method according to claim 13, wherein said marker molecule is one or more of CD96, CD20, DLL4, CD55, TIM-3, CXCR1, CD54, CD114, LGR5, CD105, CD56, CD13, CD271, CD34, CXCR4, CD26, CD117, CD10, CD146, Notch2, CD49f, CD24, ABCG2, PODXL-2, Cripto-1, CD326, CD90, CD133, SSEA1, TRA-1-81, TRA-1-60, SSEA4, SSEA3, CD151, CD340 and CD44.

15. The method according to claim 14, wherein said cancerous solid tumor cells are selected from the group consisting of one or more of osteosarcoma cells, Kaposi's sarcoma cells, melanoma cells, prostate cancer cells, glioblastoma cells, small cell lung carcinoma cells, breast cancer cells, liver cancer cells, colon cancer cells, ovarian cancer cells, renal cancer cells, gastric cancer cells, neuroblastoma cells, pancreatic cancer cells, and Hodgkin's lymphoma.

16. The method according to claim 12, wherein said diseased cells are pathogen-infected.

17. The method according to claim 16, wherein the infecting pathogen is one or more of a virus, a bacterium, a fungus, and an unicellular parasite.

18. The method according to claim 17, wherein said marker molecule is one or more of the circumsporozoite protein of P. falciparum, P. vivax, P. bergeii or P. yoelli; the M2e protein, hemagglutinin protein or neuraminidase protein of influenza virus.

19. The method according to claim 1, wherein said Diprovocim compound has a structural formula selected from the group consisting of one or more of Formulas A, B, C, D, E, F, G, H and I 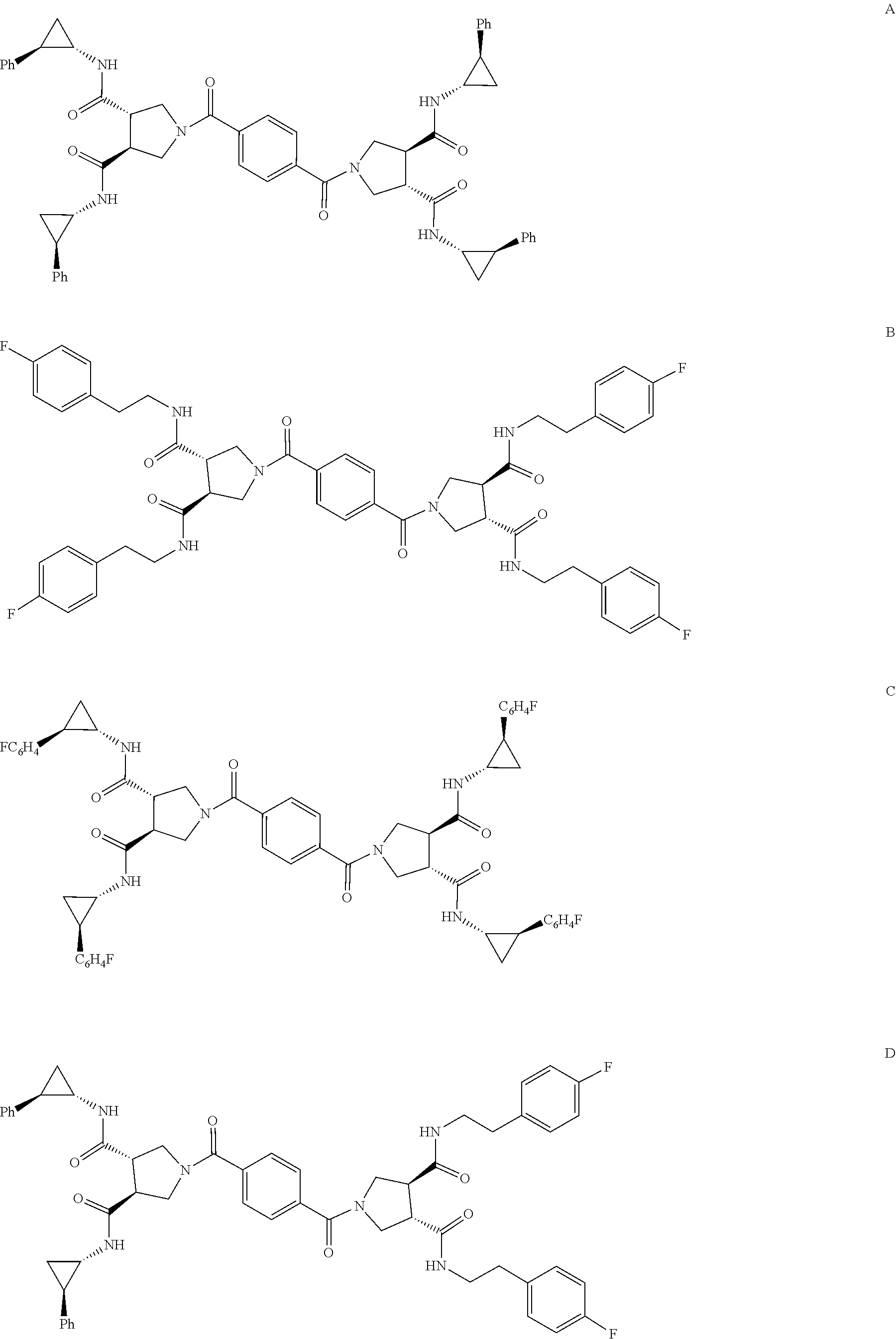 [chemical structure]


SEQUENCE LISTING (77 sequences)

SEQ ID NO: 1 - protein, 16 aa, Plasmodium falciparum
NANPNANPNANPNANP

SEQ ID NO: 2 - protein, 24 aa, Plasmodium falciparum
NANPNVDPNANPNANPNANPNVDP

SEQ ID NO: 3 - protein, 20 aa, Plasmodium falciparum
NANPNVDPNANPNANPNANP

SEQ ID NO: 4 - protein, 20 aa, Plasmodium falciparum
NANPNANPNANPNVDPNANP

SEQ ID NO: 5 - protein, 28 aa, Plasmodium falciparum
NANPNVDPNANPNANPNANPNVDPNANP

SEQ ID NO: 6 - protein, 20 aa, Plasmodium falciparum
NPNVDPNANPNANPNANPNV

SEQ ID NO: 7 - protein, 22 aa, Plasmodium falciparum
NPNVDPNANPNANPNANPNVDP

SEQ ID NO: 8 - protein, 24 aa, Plasmodium falciparum
NPNVDPNANPNANPNANPNVDPNA

SEQ ID NO: 9 - protein, 18 aa, Plasmodium falciparum
NVDPNANPNANPNANPNV

SEQ ID NO: 10 - protein, 20 aa, Plasmodium falciparum
NVDPNANPNANPNANPNVDP

SEQ ID NO: 11 - protein, 22 aa, Plasmodium falciparum
NVDPNANPNANPNANPNVDPNA

SEQ ID NO: 12 - protein, 16 aa, Plasmodium falciparum
DPNANPNANPNANPNV

SEQ ID NO: 13 - protein, 18 aa, Plasmodium falciparum
DPNANPNANPNANPNVDP

SEQ ID NO: 14 - protein, 20 aa, Plasmodium falciparum
DPNANPNANPNANPNVDPNA

SEQ ID NO: 15 - protein, 18 aa, Plasmodium vivax
DRAAGQPAGDRADGQPAG

SEQ ID NO: 16 - protein, 36 aa, Plasmodium vivax
ANGAGNQPGANGAGDQPGANGADNQPGANGADDQPG

SEQ ID NO: 17 - protein, 18 aa, Plasmodium vivax
ANGAGNQPGANGAGDQPG

SEQ ID NO: 18 - protein, 18 aa, Plasmodium vivax
ANGADNQPGANGADDQPG

SEQ ID NO: 19 - protein, 18 aa, Plasmodium vivax
ANGAGNQPGANGADNQPG

SEQ ID NO: 20 - protein, 18 aa, Plasmodium vivax
ANGADNQPGANGADDQPG

SEQ ID NO: 21 - protein, 22 aa, Plasmodium vivax
APGANQEGGAAAPGANQEGGAA

SEQ ID NO: 22 - protein, 16 aa, Plasmodium berghei
DPPPPNPNDPPPPNPN

SEQ ID NO: 23 - protein, 24 aa, Plasmodium yoelii
QGPGAPQGPGAPQGPGAPQGPGAP

SEQ ID NO: 24 - protein, 22 aa, Plasmodium falciparum
GIEYLNKIQNSLSTEWSPCSVT

SEQ ID NO: 25 - protein, 19 aa, Plasmodium vivax
YLDKVRATVGTEWTPCSVT

SEQ ID NO: 26 - protein, 20 aa, Plasmodium yoelii
EFVKQISSQLTEEWSQCSVT

SEQ ID NO: 27 - protein, 23 aa, Influenza A virus
SLLTEVETPIRNEWGCRCNGSSD

SEQ ID NO: 28 - protein, 23 aa, Influenza A virus
SLLTEVETPIRNEWGCRCNDSSD

SEQ ID NO: 29 - protein, 23 aa, Influenza A virus
SLLTEVETPIRNEWGARANDSSD

SEQ ID NO: 30 - protein, 23 aa, Influenza A virus
SLLTEVETPIRNEWGSRSNDSSD

SEQ ID NO: 31 - protein, 23 aa, Influenza A virus
SLLTEVETPIRNEWGSRCNDSSD

SEQ ID NO: 32 - protein, 23 aa, Influenza A virus
SLLTEVETPIRNEWGCRSNDSSD

SEQ ID NO: 33 - protein, 23 aa, Influenza A virus
SLLTEVETPIRNEWGCRANDSSD

SEQ ID NO: 34 - protein, 23 aa, Influenza A virus
SLLTEVETPIRNEWGARCNDSSD

SEQ ID NO: 35 - protein, 24 aa, Influenza A virus
MSLLTEVETPIRNEWGCRCNDSSD

SEQ ID NO: 36 - protein, 24 aa, Influenza A virus
MSLLTEVETPIRNEWGSRSNDSSD

SEQ ID NO: 37 - protein, 35 aa, Influenza A virus
MGISLLTEVETPIRNEWGCRCNDSSDELLGWLWGI

SEQ ID NO: 38 - protein, 24 aa, Influenza A virus
MSLLTEVETPIRNEWGARANDSSD

SEQ ID NO: 39 - protein, 24 aa, Influenza A virus
MSLLTEVETPIRNEWGCRANDSSD

SEQ ID NO: 40 - protein, 24 aa, Influenza A virus
MSLLTEVETPIRNEWGARCNDSSD

SEQ ID NO: 41 - protein, 24 aa, Influenza A virus
MSLLTEVETPIRNEWGCRSNDSSD

SEQ ID NO: 42 - protein, 24 aa, Influenza A virus
MSLLTEVETPIRNEWGSRCNDSSD

SEQ ID NO: 43 - protein, 46 aa, Influenza A virus
SLLTEVETPIRNEWGSRSNDSSDSLLTEVETPIRNEWGSRSNDSSD

SEQ ID NO: 44 - protein, 69 aa, Influenza A virus
SLLTEVETPIRNEWGSRSNDSSDSLLTEVETPIRNEWGSRSNDSSDSLLTEVETPIRNEWGCRCNDSSD

SEQ ID NO: 45 - protein, 46 aa, Influenza A virus
SLLTEVETPIRNEWGARANDSSDSLLTEVETPIRNEWGCRCNDSSD

SEQ ID NO: 46 - protein, 69 aa, Influenza A virus
SLLTEVETPIRNEWGARANDSSDSLLTEVETPIRNEWGARANDSSDSLLTEVETPIRNEWGCRCNDSSD

SEQ ID NO: 47 - protein, 19 aa, Influenza A virus
EVETPIRNEWGSRCNDSSD

SEQ ID NO: 48 - protein, 38 aa, Influenza A virus
EVETPIRNEWGSRCNDSSDEVETPIRNEWGSRCNDSSD

SEQ ID NO: 49 - protein, 57 aa, Influenza A virus
EVETPIRNEWGSRCNDSSDEVETPIRNEWGSRCNDSSDEVETPIRNEWGCRCNDSSD

SEQ ID NO: 50 - protein, 92 aa, Influenza A virus
SLLTEVETPIRNEWGSRSNDSSDSLLTEVETPIRNEWGSRSNDSSDSLLTEVETPIRNEWGSRSNDSSDSLLTEVETPIRNEWGCRCNDSSD

SEQ ID NO: 51 - protein, 115 aa, Influenza A virus
SLLTEVETPIRNEWGSRSNDSSDSLLTEVETPIRNEWGSRSNDSSDSLLTEVETPIRNEWGSRSNDSSDSLLTEVETPIRNEWGSRSNDSSDSLLTEVETPIRNEWGCRCNDSSD

SEQ ID NO: 52 - protein, 23 aa, Influenza A virus
SLLTEVETPTRNEWGCRCNDSSD

SEQ ID NO: 53 - protein, 23 aa, Influenza A virus
SLLTEVETPTRNGWGCRCNDSSD

SEQ ID NO: 54 - protein, 23 aa, Influenza A virus
SLLTEVETPIRNEWECRCNGSSD

SEQ ID NO: 55 - protein, 23 aa, Influenza A virus
SLLTEVETPTKNEWECRCNDSSD

SEQ ID NO: 56 - protein, 23 aa, Influenza A virus
SLLTEVETPTRNGWECKCSDSSD

SEQ ID NO: 57 - protein, 23 aa, Influenza A virus
SLLTEVDTLTRNGWGCRCSDSSD

SEQ ID NO: 58 - protein, 23 aa, Influenza A virus
SLLTEVETLTRNGWECKCRDSSD

SEQ ID NO: 59 - protein, 17 aa, Influenza B virus
NNATFNYTNVNPISHIR

SEQ ID NO: 60 - protein, 28 aa, Hepatitis B virus
FPGSSTTSTGPCRTCMTTAQGTSMYPSC

SEQ ID NO: 61 - protein, 13 aa, Hepatitis B virus
MTTAQGTSMYPSC

SEQ ID NO: 62 - protein, 28 aa, Hepatitis B virus
IPGSTTTSTGPCKTCTTPAQGNSMFPSC

SEQ ID NO: 63 - protein, 13 aa, Hepatitis B virus
TTPAQGNSMFPSC

SEQ ID NO: 64 - protein, 31 aa, Hepatitis B virus
CPLIPGSTTTSTGPCKTCTTPAQGNSMFPSC

SEQ ID NO: 65 - protein, 34 aa, Hepatitis B virus
CLGQNSQSPTSNHSPTSCPPTCPGYRWMCLRRFI

SEQ ID NO: 66 - protein, 15 aa, Hepatitis B virus
SLNFLGGTTVCLGQN

SEQ ID NO: 67 - protein, 6 aa, Hepatitis B virus
VCLGQN

SEQ ID NO: 68 - protein, 19 aa, Hepatitis B virus
LIFLLVLLDYQGMLPVCPL

SEQ ID NO: 69 - protein, 15 aa, Human papillomavirus type 16
MADPAGTNGEEGTGC

SEQ ID NO: 70 - protein, 16 aa, Human papillomavirus type 16
HEDEDKENDGDSLPTC

SEQ ID NO: 71 - protein, 11 aa, Human papillomavirus type 16
RPFKSNKSTCC

SEQ ID NO: 72 - protein, 15 aa, Human papillomavirus type 16
CCDWCIAAFGLTPSI

SEQ ID NO: 73 - protein, 19 aa, Human papillomavirus type 16
TYDSEWQRDQFLSQVKIPC

SEQ ID NO: 74 - protein, 19 aa, Human papillomavirus type 16
HKSAIVTLTYDSEWQRDQC

SEQ ID NO: 75 - protein, 16 aa, Human papillomavirus type 16
CINCQKPLCPEEKQRH

SEQ ID NO: 76 - RNA, 20 nt, Unknown
  Tlr1 small base-pairing guide DNA
caaaccgaucguagugcuga

SEQ ID NO: 77 - protein, 8 aa, Gallus gallus
SIINFEKL